SEQUENCE LISTING (67 sequences)

SEQ ID NO: 1 - protein, 619 aa, Arachis hypogea
  Ara h1
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRRALILTRAMHSESHPFHFLHLDDMTPELRVAKSHAVNTPGQFEDFFPASSRDQSSYLQGFSRNTLEAAFNAEFNEIRRVLLEENAGGEQEERGQRRWSTRSSENNEGVIVEVSKEHVEELTKHAKSVSKKGSEEEGDITNPINLREGEPDLSDNFGRLFEVKPDKKNPQLQDLDMMLTCVEIKEGALMLPHFNSKAMVIVVINKGTGNLELVAVRKEQQQRGRREQEWEEEEEDEEEEGSNREVRRYTARLKEGDVFIMPAAHPVAINASSELHLLGFGINAENNHRIFLAGDKDNVVDQIEKQAKDLAFPGSGEQVEKLIKNQRESHFVSARPQSQSPSSPEKEDQEEENQGGKGPLLSILKAFN

SEQ ID NO: 2 - protein, 172 aa, Arachis hypogea
  Ara h2
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 3 - protein, 512 aa, Arachis hypogea
  Ara h3
MAKLLALSLCFCVLVLGASSVTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNGIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVA

SEQ ID NO: 4 - protein, 15 aa, Arachis hypogea
  ara h 1.008 peptide
ATHAKSSPYQKKTEN

SEQ ID NO: 5 - protein, 15 aa, Arachis hypogea
  ara h 1.015 peptide
LQSCQQEPDDLKQKA

SEQ ID NO: 6 - protein, 15 aa, Arachis hypogea
  ara h 1.021 peptide
RCTKLEYDPRCVYDP

SEQ ID NO: 7 - protein, 15 aa, Arachis hypogea
  ara h 1.022 peptide
KLEYDPRCVYDPRGH

SEQ ID NO: 8 - protein, 15 aa, Arachis hypogea
  ara h 1.025 peptide
YDPRGHTGTTNQRSP

SEQ ID NO: 9 - protein, 15 aa, Arachis hypogea
  ara h 1.029 peptide
RSPPGERTRGRQPGD

SEQ ID NO: 10 - protein, 15 aa, Arachis hypogea
  ara h 1.030 peptide
PGERTRGRQPGDYDD

SEQ ID NO: 11 - protein, 15 aa, Arachis hypogea
  ara h 1.033 peptide
PGDYDDDRRQPRREE

SEQ ID NO: 12 - protein, 15 aa, Arachis hypogea
  ara h 1.035 peptide
DRRQPRREEGGRWGP

SEQ ID NO: 13 - protein, 15 aa, Arachis hypogea
  ara h 1.040 peptide
AGPREREREEDWRQP

SEQ ID NO: 14 - protein, 15 aa, Arachis hypogea
  ara h 1.041 peptide
REREREEDWRQPRED

SEQ ID NO: 15 - protein, 15 aa, Arachis hypogea
  ara h 1.044 peptide
RQPREDWRRPSHQQP

SEQ ID NO: 16 - protein, 15 aa, Arachis hypogea
  ara h 1.045 peptide
REDWRRPSHQQPRKI

SEQ ID NO: 17 - protein, 15 aa, Arachis hypogea
  ara h 1.047 peptide
PSHQQPRKIRPEGRE

SEQ ID NO: 18 - protein, 15 aa, Arachis hypogea
  ara h 1.050 peptide
RPEGREGEQEWGTPG

SEQ ID NO: 19 - protein, 15 aa, Arachis hypogea
  ara h 1.056 peptide
REETSRNNPFYFPSR

SEQ ID NO: 20 - protein, 15 aa, Arachis hypogea
  ara h 1.058 peptide
NNPFYFPSRRFSTRY

SEQ ID NO: 21 - protein, 15 aa, Arachis hypogea
  ara h 1.090 peptide
SGFISYILNRHDNQN

SEQ ID NO: 22 - protein, 15 aa, Arachis hypogea
  ara h 1.097 peptide
SMPVNTPGQFEDFFP

SEQ ID NO: 23 - protein, 15 aa, Arachis hypogea
  ara h 1.103 peptide
RDQSSYLQGFSRNTL

SEQ ID NO: 24 - protein, 15 aa, Arachis hypogea
  ara h 1.130 peptide
SEEEGDITNPINLRE

SEQ ID NO: 25 - protein, 15 aa, Arachis hypogea
  ara h 1.131 peptide
EGDITNPINLREGEP

SEQ ID NO: 26 - protein, 15 aa, Arachis hypogea
  ara h 1.137 peptide
NNFGKLFEVKPDKKN

SEQ ID NO: 27 - protein, 15 aa, Arachis hypogea
  ara h 1.167 peptide
RYTARLKEGDVFIMP

SEQ ID NO: 28 - protein, 15 aa, Arachis hypogea
  ara h 1.170 peptide
DVFIMPAAHPVAINA

SEQ ID NO: 29 - protein, 15 aa, Arachis hypogea
  ara h 1.173 peptide
PVAINASSELHLLGF

SEQ ID NO: 30 - protein, 15 aa, Arachis hypogea
  ara h 1.176 peptide
LHLLGFGINAENNHR

SEQ ID NO: 31 - protein, 15 aa, Arachis hypogea
  ara h 1.179 peptide
AENNHRIFLAGDKDN

SEQ ID NO: 32 - protein, 15 aa, Arachis hypogea
  ara h 1.180 peptide
NHRIFLAGDKDNVID

SEQ ID NO: 33 - protein, 15 aa, Arachis hypogea
  ara h 1.184 peptide
VIDQIEKQAKDLAFP

SEQ ID NO: 34 - protein, 15 aa, Arachis hypogea
  ara h 1.186 peptide
KQAKDLAFPGSGEQV

SEQ ID NO: 35 - protein, 15 aa, Arachis hypogea
  ara h 1.187 peptide
KDLAFPGSGEQVEKL

SEQ ID NO: 36 - protein, 15 aa, Arachis hypogea
  ara h 1.194 peptide
SHFVSARPQSQSQSP

SEQ ID NO: 37 - protein, 15 aa, Arachis hypogea
  ara h 1.203 peptide
QEEENQGGKGPLLSI

SEQ ID NO: 38 - protein, 15 aa, Arachis hypogea
  ara h 2.005 peptide
AAHASARQQWELQGD

SEQ ID NO: 39 - protein, 15 aa, Arachis hypogea
  ara h 2.008 peptide
WELQGDRRCQSQLER

SEQ ID NO: 40 - protein, 15 aa, Arachis hypogea
  ara h 2.010 peptide
RRCQSQLERANLRPC

SEQ ID NO: 41 - protein, 15 aa, Arachis hypogea
  ara h 2.014 peptide
RPCEQHLMQKIQRDE

SEQ ID NO: 42 - protein, 15 aa, Arachis hypogea
  ara h 2.017 peptide
KIQRDEDSYERDPYS

SEQ ID NO: 43 - protein, 15 aa, Arachis hypogea
  ara h 2.018 peptide
RDEDSYERDPYSPSQ

SEQ ID NO: 44 - protein, 15 aa, Arachis hypogea
  ara h 2.019 peptide
DSYERDPYSPSQDPY

SEQ ID NO: 45 - protein, 15 aa, Arachis hypogea
  ara h 2.021 peptide
PYSPSQDPYSPSPYD

SEQ ID NO: 46 - protein, 15 aa, Arachis hypogea
  ara h 2.030 peptide
CCNELNEFENNQRCM

SEQ ID NO: 47 - protein, 15 aa, Arachis hypogea
  ara h 2.031 peptide
ELNEFENNQRCMCEA

SEQ ID NO: 48 - protein, 15 aa, Arachis hypogea
  ara h 2.036 peptide
LQQIMENQSDRLQGR

SEQ ID NO: 49 - protein, 15 aa, Arachis hypogea
  ara h 2.037 peptide
IMENQSDRLQGRQQE

SEQ ID NO: 50 - protein, 15 aa, Arachis hypogea
  ara h 2.038 peptide
NQSDRLQGRQQEQQF

SEQ ID NO: 51 - protein, 15 aa, Arachis hypogea
  ara h 2.040 peptide
QGRQQEQQFKRELRN

SEQ ID NO: 52 - protein, 15 aa, Arachis hypogea
  ara h 2.043 peptide
KRELRNLPQQCGLRA

SEQ ID NO: 53 - protein, 15 aa, Arachis hypogea
  ara h 2.045 peptide
LPQQCGLRAPQRCDL

SEQ ID NO: 54 - protein, 15 aa, Arachis hypogea
  ara h 3.018 peptide
LRRNALRRPFYSNAP

SEQ ID NO: 55 - protein, 15 aa, Arachis hypogea
  ara h 3.030 peptide
HYEEPHTQGRRSQSQ

SEQ ID NO: 56 - protein, 15 aa, Arachis hypogea
  ara h 3.031 peptide
EPHTQGRRSQSQRPP

SEQ ID NO: 57 - protein, 15 aa, Arachis hypogea
  ara h 3.037 peptide
QGEDQSQQQRDSHQK

SEQ ID NO: 58 - protein, 15 aa, Arachis hypogea
  ara h 3.060 peptide
NTEQEFLRYQQQSRQ

SEQ ID NO: 59 - protein, 15 aa, Arachis hypogea
  ara h 3.068 peptide
PYSPQSQPRQEEREF

SEQ ID NO: 60 - protein, 15 aa, Arachis hypogea
  ara h 3.079 peptide
EGGNIFSGFTPEFLE

SEQ ID NO: 61 - protein, 15 aa, Arachis hypogea
  ara h 3.080 peptide
NIFSGFTPEFLEQAF

SEQ ID NO: 62 - protein, 15 aa, Arachis hypogea
  ara h 3.092 peptide
AIVTVRGGLRILSPD

SEQ ID NO: 63 - protein, 15 aa, Arachis hypogea
  ara h 3.093 peptide
TVRGGLRILSPDRKR

SEQ ID NO: 64 - protein, 15 aa, Arachis hypogea
  ara h 3.100 peptide
EYDEDEYEYDEEDRR

SEQ ID NO: 65 - protein, 15 aa, Arachis hypogea
  ara h 3.102 peptide
YEYDEEDRRRGRGSR

SEQ ID NO: 66 - protein, 15 aa, Arachis hypogea
  ara h 3.152 peptide
IANLAGENSVIDNLP

SEQ ID NO: 67 - protein, 15 aa, Arachis hypogea
  ara h 3.162 peptide
RQLKNNNPFKFFVPP

(19) United States
(12) Patent Application Publication
(10) Pub. No.: US 2019/0359660 A1
(43) Pub. Date: Nov. 28, 2019

(54) Peptides And Methods For Detecting Peanut Allergies

(71) Applicants: AllerGenis LLC, Hatfield, PA (US); Icahn School of Medicine at Mount Sinai, New York, NY (US)

(72) Inventors: Robert C. Getts, Hatfield, PA (US); Paul Kearney, Hatfield, PA (US); Hugh A. Sampson, New York, NY (US); Mayte Suarez-Farinas, New York, NY (US)

(21) Appl. No.: 16/417,946

(22) Filed: May 21, 2019

Related U.S. Application Data
(60) Provisional application No. 62/674,656, filed on May 22, 2018.

(51) Int. Cl. C07K 14/415; G01N 33/68; A61P 37/08; A61K 39/35

(52) U.S. Cl. CPC C07K 14/415; A61K 39/35; A61P 37/08; G01N 33/6893

(57) ABSTRACT

The present disclosure provides peptide biomarkers, including methods and kits employing the same, for diagnosis of peanut allergy, and tolerance thereto, and for determining whether an allergic subject is likely to outgrow the allergy.

### FIELD

The present disclosure is directed, in part, to peptide biomarkers, including methods and kits employing the same, for diagnosis of peanut allergy and for determining whether an allergic subject is likely to outgrow the allergy.

### BACKGROUND

Food allergies are a common problem among adults and children, and symptoms may range from mild oral pruritus to potentially life-threatening anaphylactic shock. Food allergies are currently diagnosed by skin prick testing or oral provocation, and measurement of serum levels of specific IgE and, in some cases, other serum antibodies, such as IgG4. Although these tests indicate the likelihood of clinical reactivity, they do not distinguish the different phenotypes of food allergy or provide prognostic information. Current allergy tests also involve some level of risk to the patient. The relationship between current IgE testing and the actual clinical sensitivity of the patient is a weak one that is usually defined as a combination of reaction severity and the amount of allergen that provokes a reaction. Another limitation of current testing is the inability to determine whether or not pediatric patients will outgrow the allergy during childhood. In this case there is a positive but weak correlation between specific IgE level and the duration of clinical allergy.

More recently, it has been suggested that clinical reactivity to food allergens may correlate better with allergen-specific IgE on the epitope recognition level. It has been reported that patients with persistent or more severe allergic reactions recognize larger numbers of IgE epitopes, suggesting epitope mapping as an additional tool for allergy diagnosis and prediction. Spot membrane-based immunoassays have been used for epitope mapping. In this system, peptides are synthesized on the membrane and incubated with the patient's sera. The process requires a large number of peptides and is, therefore, error prone, time consuming, labor intensive, and expensive. Immunoassays in this format also require a large volume of patient serum.

The marked heterogeneity of clinical presentations for food allergy poses a challenge to successful management and treatment, and therefore precision medicine strategies are highly relevant to improve prevention, manage current cases and initiate new therapy in food allergy. Sensitive and specific biomarkers for determination of food allergy endotypes, risk of developing allergies, reaction severity, and prognosis with treatment are essential components in the path toward precision medicine (Sicherer et al., J. Allergy Clin. Immunol., 2015, 135, 357-67). In the past decade, there have been a number of studies evaluating the efficacy of oral immunotherapy (OIT) for the treatment of persistent food allergies (Wood et al., J. Allergy Clin. Immunol., 2016, 137, 1103-1110). In peanut allergy, OIT has been shown to have acceptable safety profile and demonstrated clinical benefit (Bird et al., J. Allergy Clin. Immunol. Pract., 2017, 5, 335-344). Despite the improvement in clinical reactivity, OIT has been associated with significant adverse effects, with some experiencing anaphylaxis and 15% to 20% forced to discontinue therapy because of adverse reactions (Bird et al., J. Allergy Clin. Immunol. Pract., 2017; Keet Et al., J. Allergy Clin. Immunol., 2012, 129, 448-455; Longo et al., J. Allergy Clin. Immunol., 2008, 121, 343-7; Meglio et al., Pediatr. Allergy Immunol., 2008, 19, 412-419; Skripak et al., J. Allergy Clin. Immunol., 2008, 122, 1154-60; Staden et al., Allergy, 2007, 62, 1261-1269). In addition to adverse reactions, the response to OIT is typically not sustained once therapy is discontinued, i.e. patients are temporarily desensitized to allergens but do not achieve tolerance (Wood et al., J. Allergy Clin. Immunol., 2016, 137, 1103-1110; Burks et al., N. Engl. J. Med., 2012, 367, 233-243; Burks et al., J. Allergy Clin. Immunol., 2008, 121, 1344-1350; Burks, Arb. Paul Ehrlich Inst. Bundesinstitut Impfstoffe Biomed Arzneim Langen Hess, 2013, 97, 122-123; Gorelik et al., J. Allergy Clin. Immunol., 2015, 135, 1283-1292; and Keet et al., J. Allergy Clin. Immunol., 2013, 132, 737-739). However, it is clear that progress is being made and new food allergy therapies are close to FDA approval. These therapeutic approaches will benefit from a diagnostic and prognostic test which will help patients and their doctors understand the severity of the disease upon entry into therapy, monitor a patient while on therapy to assess progress or onset of an adverse reaction before it occurs, and track patient status once treatment is discontinued.

The production of IgE antibodies against peanut proteins is central to the pathogenesis of peanut allergy. Although predictive curves have been generated to identify peanut specific IgE concentrations which are 95% predictive of clinical reactivity, peanut-IgE is poorly predictive at lower IgE levels, and at higher levels the readout is only binary and is therefore difficult to use to help assess the safety or efficacy of therapy. This may be due to measurement of IgE antibodies against components of peanut which are not clinically relevant. IgE against Ara h 2 predicts clinical reactivity to peanut (Lieberman et al., J. Allergy Clin. Immunol. Pract., 2013, 1, 75-82) but there is a great deal of clinical heterogeneity across individuals with similar levels of Ara h 2. Peptide microarrays comprised of overlapping peptides covering the entire sequential epitope repertoire of major allergens have been developed to measure the epitope-specific immunoglobulin response (Lin et al., J. Allergy Clin. Immunol., 2009, 124, 315-22; and Lin et al., J. Allergy Clin. Immunol., 2012, 129, 1321-1328). The number of peanut epitopes in Ara h 1, 2 and 3 which bind to IgE is predictive of reaction severity (Flinterman et al., J. Allergy Clin. Immunol., 2008, 121, 737-743). As a component-resolved diagnostic methodology (ImmunoCAP), the presence of sIgE to peanut, Ara h1, Ara h2, and Ara h3 is indicative of a “true” peanut allergy and a high risk of severe reactions (e.g., levels of sIgE≥0.35 kU_{A}/L show 75-95% PPV, 90% NPV in diagnosing allergy; Klemans et al., J. Allergy Clin. Immunol., 2013, 131, 157-163).

### SUMMARY

Historically it was thought that people develop allergies to a specific protein, but the chemistry and immune response is much more granular. Studying IgE to whole peanut extract or even components does not provide the specificity of the true response at the molecular level. Proteins contain various epitopes along their structures which are the specific targets to which an antibody attaches itself during an allergic response. Therefore, a higher resolution allergy test panel can identify the specific epitopes within a protein responsible for an allergic response in a patient. The methods described herein are partly based on the premise that by subdividing the proteins found in peanuts into shorter components (epitopes) that make up the whole protein, the antibody reactivity can be stratified based on how they bind to each epitope, thereby establishing an epitope binding pattern for each patient and enabling a more accurate and predictive diagnosis.

As described herein, the methods subdivide the proteins found in peanuts into smaller peptides, or compounds consisting of two or more amino acids. The mapped peptides are then separately coupled to beads to allow for high-throughput analysis and epitope binding assessment. The reactivity of the patient's IgE response, those antibodies in the bloodstream which act as “the match that lights the fire” of an allergic immune response, is examined. In the reaction, antibodies attach to peptide coupled beads and the methods are designed to isolate and determine individualized reactions per peptide so clinicians can obtain a more accurate and complete picture of a patient's allergy profile. The results are “mapped” with individual peptide results, creating a therapy response profile used to classify patients.

The present disclosure provides methods for diagnosing a peanut allergy, and/or severity of a peanut allergy, in a subject comprising: contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptides are coupled to a solid support, to form one or more AAI-peptide-solid support complexes; binding an AAI-specific labeling reagent to the AAI-peptide-solid support complex; and detecting binding of the AAI-specific labeling reagent to each AAI-peptide-solid support complex to identify one or more peanut peptides bound to the AAI in the serum or plasma of the subject; wherein recognition of at least one peanut peptide by an AAI in the serum or plasma of the subject indicates that the subject is allergic to peanuts.

The present disclosure also provides methods for detecting development of clinical tolerance to peanuts in a subject that is allergic to peanuts comprising: contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptides are coupled to a solid support, to form one or more AAI-peptide-solid support complexes; binding an AAI-specific labeling reagent to the AAI-peptide-solid support complex; detecting binding of the AAI-specific labeling reagent to each AAI-peptide-solid support complex to identify one or more peanut peptides bound to the AAI in the serum or plasma of the subject; and comparing the identified one or more peanut peptides bound to the AAI in the serum or plasma of the subject, or the concentration of the AAI in the serum or plasma of the subject, with a previously identified panel of one or more peanut peptides bound to the AAI in the serum or plasma of the subject, or a previous concentration of the AAI in the serum or plasma of the subject; wherein development of clinical tolerance to peanuts is indicated when: the subsequent number of peanut peptides recognized by IgE AAI in the serum or plasma of the subject, and/or the subsequent concentration of AAI IgE in the serum or plasma of the subject, is less than the previously identified number of peanut peptides recognized by AAI IgE in the serum or plasma of the subject, and/or less than the previous concentration of AAI IgE in the serum or plasma of the subject; and/or the subsequent number of peanut peptides recognized by IgG4 AAI in the serum or plasma of the subject, and/or the subsequent concentration of AAI IgG4 in the serum or plasma of the subject, is greater than the previously identified number of peanut peptides recognized by AAI IgG4 in the serum or plasma of the subject, and/or greater than the previous concentration of AAI IgG4 in the serum or plasma of the subject.

The present disclosure also provides methods for detecting an increase in intensity of allergy or adverse event during treatment of allergy to peanuts over time in a subject that is allergic to peanuts comprising: contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptide are coupled to a solid support, to form one or more AAI-peptide-solid support complexes, and wherein the one or more peanut peptides is selected from peptides having at least 3 contiguous amino acids from positions 8 to 66 of ara h 1 allergen, peptides having at least 3 contiguous amino acids from positions 103 to 152 of ara h 1 allergen, peptides having at least 3 contiguous amino acids from positions 176 to 195 of ara h 1 allergen, peptides having at least 3 contiguous amino acids from positions 5 to 40 of ara h 2 allergen, peptides having at least 3 contiguous amino acids from positions 93 to 115 of ara h 3 allergen, peptides having at least 3 contiguous amino acids from positions 30 to 75 of ara h 3 allergen, and/or peptides having at least 3 contiguous amino acids from positions 152 to 167 of ara h 3 allergen; binding an AAI-specific labeling reagent to the AAI-peptide-solid support complex; detecting binding of the AAI-specific labeling reagent to each AAI-peptide-solid support complex to identify one or more peanut peptides bound to the AAI in the serum or plasma of the subject; and comparing the identified one or more peanut peptides bound to the AAI in the serum or plasma of the subject, or the concentration of the AAI in the serum or plasma of the subject, with a previously identified panel of one or more peanut peptides bound to the AAI in the serum of the subject, or a previous concentration of the AAI in the serum or plasma of the subject; wherein an increased intensity of the allergic response to peanuts is indicated when the subsequent number or pattern of reactivity of peanut peptides recognized by AAI in the serum or plasma of the subject, or the subsequent concentration of AAI in the serum or plasma of the subject, is greater than the previously identified number or pattern of reactivity of peanut peptides recognized by AAI in the serum or plasma of the subject, or greater than the previous concentration of AAI in the serum or plasma of the subject.

The present disclosure also provides methods of sensitizing an infant to one or more peanut allergens to induce tolerance or non-allergy to peanuts comprising administering one or more peanut peptides to the infant, wherein the one or more peanut peptides are derived from ara h 1 allergen (SEQ ID NO:1), ara h 2 allergen (SEQ ID NO:2), and/or ara h 3 allergen (SEQ ID NO:3).

The present disclosure also provides sets of allergenic epitope-containing peanut peptides comprising a plurality of peanut peptides comprising at least two peptides derived from ara h 1 allergen (SEQ ID NO:1), ara h 2 allergen (SEQ ID NO:2), and/or ara h 3 allergen (SEQ ID NO:3).

The present disclosure also provides kits comprising: one or more allergenic epitope-containing peanut peptides derived from ara h 1 allergen (SEQ ID NO:1), ara h 2 allergen (SEQ ID NO:2), and/or ara h 3 allergen (SEQ ID NO:3), wherein each peanut peptide is coupled to a solid support; and an allergy associated immunoglobulin (AAI)-specific labeling reagent; packaged together and including instructions for use.

### BRIEF DESCRIPTION OF THE DRAWINGS

FIG. 1 shows a representative IgE response for IgE epitope differences between Avoiders and Consumers across study visits compared to the baseline visit at the beginning of the study (V12-V1, V30-V1, and V60-V1).

FIG. 2 shows a representative IgG4 response for IgG4 epitope differences between Avoiders and Consumers across study visits compared to the baseline visit at the beginning of the study (V12-V1, V30-V1, and V60-V1).

FIG. 3 shows a representative IgE response between groups as a basis of changes per visit.

FIG. 4 shows a representative IgG4 response changing at Visit 60.

FIG. 5 shows a representative IgG4 response in the Avoider group at V60.

FIG. 6 shows a representative IgG4 epitope expansion in Consumers who were sensitized compared to Avoiders.

FIG. 7 shows a representative results of 64 peanut allergic epitope assay and EB scores at 1, 2.5, and 5 years of age compared with their baseline visit.

FIG. 8 shows representative results of epitope profiles.

FIG. 9 shows representative epitope model in training results.

FIG. 10 shows a representative epitope model in testing results.

FIG. 11 shows representative results of a comparison of epitopes model and ImmunoCAP.

FIG. 12 shows a representative AUC in CV for epitopes and epitopes+sIgE.

FIG. 13 shows representative results of the accuracy of the epitopes model in testing.

FIG. 14 shows a ROC plot of performance in CoFar2.

### DESCRIPTION OF EMBODIMENTS

The terminology used herein is for the purpose of describing particular embodiments only and is not intended to be limiting.

Before describing several exemplary embodiments, it is to be understood that the embodiments is not limited to the details of construction or process steps set forth in the following description. The embodiments described herein are capable of modifications and of being practiced or being carried out in various ways.

Reference throughout the present disclosure to “some embodiments,” or derivations thereof, means that a particular feature, structure, material, or characteristic described in connection with the embodiment is included in at least one embodiment. Thus, the appearances of the phrases such as “in some embodiments,” in various places throughout the present disclosure is not necessarily referring to the same embodiment, but can generally be attributed to any other embodiment. Furthermore, the particular features, structures, materials, or characteristics may be combined in any suitable manner in one or more embodiments.

As used herein, the terms “allergy associated immunoglobulin” and “AAI” refer to immunoglobulins in sera that mediate hypersensitivity to peanut allergens. These include one or more of IgE, IgA, IgM, and IgG (including IgG4).

As used herein, the terms “reactive”, “reactivity”, “recognize” and the like refer to the ability of an allergy associated immunoglobulin to bind to an allergenic epitope containing peptide. The level of reactivity indicates the concentration of AAI in the serum or plasma, with high reactivity associated with higher AAI concentrations and lower reactivity associated with lower AAI concentrations. The relative AAI concentration (i.e., the relative serum or plasma reactivity) is determined by the amount of signal detected in the assay. The level of reactivity of AAI to allergenic epitope containing peptides also indicates the intensity of the allergic response (i.e., higher reactivity is associated with a more intense allergic reaction).

As used herein, the term “clinical tolerance” refers to immunological tolerance to a peanut allergen that is developed by an allergic subject as a result of exposure to the allergen (i.e., tolerance developed as a result of immunotherapy).

As used herein, the term “natural tolerance” refers to immunological tolerance to a peanut allergen that is developed by an allergic subject as a biochemical process over time, either as a result of natural exposure to the allergen during a lifetime or in the absence of exposure.

The present disclosure provides sets of allergenic epitope-containing peanut peptides comprising a plurality of peanut peptides comprising at least two peptides derived from ara h 1 allergen (SEQ ID NO:1), ara h 2 allergen (SEQ ID NO:2), and/or ara h 3 allergen (SEQ ID NO:3).

In some embodiments, the plurality of peanut peptides is selected from: peptides having at least 3 contiguous amino acids from positions 8 to 66 of ara h 1 allergen; peptides having at least 3 contiguous amino acids from positions 103 to 152 of ara h 1 allergen; peptides having at least 3 contiguous amino acids from positions 176 to 195 of ara h 1 allergen; peptides having at least 3 contiguous amino acids from positions 5 to 40 of ara h 2 allergen; peptides having at least 3 contiguous amino acids from positions 93 to 115 of ara h 3 allergen; peptides having at least 3 contiguous amino acids from positions 30 to 75 of ara h 3 allergen; and/or peptides having at least 3 contiguous amino acids from positions 152 to 167 of ara h 3 allergen.

In some embodiments, the plurality of peanut peptides comprise peptides having an amino acid sequence selected from any one or more of SEQ ID NOs: 4-67. In some embodiments, the plurality of peanut peptides comprise peptides having an amino acid sequence selected from any one or more of SEQ ID NOs: 7, 13, 16, 17, 25, 27, 30, 36, 39, 45, 65, and 66. In some embodiments, the plurality of peanut peptides comprise peptides having an amino acid sequence selected from any one or more of SEQ ID NOs: 39, 45, and 66. In some embodiments, the plurality of peanut peptides comprise peptides having an amino acid sequence selected from any one or more of SEQ ID NOs: 5, 6, 10, 13, 14, 34, 36, 39, 40, 42, 49, 61, 65, and 66. In some embodiments, the plurality of peanut peptides comprise peptides having an amino acid sequence selected from any one or more of SEQ ID NOs: 5, 6, 9, 34, 36, 39, and 40. In some embodiments, the plurality of peanut peptides comprise peptides having an amino acid sequence selected from any one or more of SEQ ID NOs: 29, 39, 42, 44, 45, 51, and 63. In some embodiments, the plurality of peanut peptides comprise peptides having an amino acid sequence selected from any one or more of SEQ ID NOs: 7, 8, 29, 31, 39, 45, and 61. In some embodiments, the plurality of peanut peptides comprise peptides having an amino acid sequence selected from any one or more of SEQ ID NOs: 39 and 40. In some embodiments, the plurality of peanut peptides comprise a peptide having an amino acid sequence of SEQ ID NO: 39. In some embodiments, the plurality of peanut peptides comprise a peptide having an amino acid sequence of SEQ ID NO: 40.

In some embodiments, each peanut peptide comprises from about 3 amino acids to about 60 amino acids, from about 4 amino acids to about 60 amino acids, from about 6 amino acids to about 30 amino acids, from about 7 amino acids to about 20 amino acids, from about 10 amino acids to about 16 amino acids, or from about 10 amino acids to about 15 amino acids. In some embodiments, each peanut peptide comprises 15 amino acids.

In some embodiments, the plurality of peanut peptides comprises at least 2 peanut peptides, at least 3 peanut peptides, at least 5 peanut peptides, at least 10 peanut peptides, at least 15 peanut peptides, at least 20 peanut peptides, at least 25 peanut peptides, at least 30 peanut peptides, at least 35 peanut peptides, at least 40 peanut peptides, at least 45 peanut peptides, at least 50 peanut peptides, at least 55 peanut peptides, at least 60 peanut peptides, or at least 64 peanut peptides. In some embodiments, the plurality of peanut peptides comprises from about 2 to about 64 peanut peptides, from about 2 to about 60 peanut peptides, from about 2 to about 55 peanut peptides, from about 2 to about 50 peanut peptides, from about 2 to about 45 peanut peptides, from about 2 to about 40 peanut peptides, from about 2 to about 35 peanut peptides, from about 2 to about 30 peanut peptides, from about 2 to about 25 peanut peptides, from about 2 to about 20 peanut peptides, from about 2 to about 15 peanut peptides, or from about 2 to about 10 peanut peptides. In some embodiments, the plurality of peanut peptides comprises from about 2 to about 64 peanut peptides, from about 5 to about 64 peanut peptides, from about 10 to about 64 peanut peptides, from about 15 to about 64 peanut peptides, from about 20 to about 64 peanut peptides, from about 25 to about 64 peanut peptides, from about 30 to about 64 peanut peptides, from about 35 to about 64 peanut peptides, from about 40 to about 64 peanut peptides, from about 45 to about 64 peanut peptides, from about 50 to about 64 peanut peptides, or from about 55 to about 64 peanut peptides.

It is to be understood that although the allergenic epitope-containing peptides disclosed herein are described as specific embodiments having specific amino acid sequences, one skilled in the art will recognize that each such peptide may be shifted in either the N-terminal or C-terminal direction of the protein from which it is derived to obtain a related peptide sequence that still contains the relevant epitope but in which the relevant epitope is flanked by different amino acids than specified. Accordingly, in all embodiments, the allergenic epitope containing peptides can have amino acid sequences that overlap with the disclosed peptide sequences by 2, 4, 6, or 8 or more contiguous amino acids.

It will also be recognized that analysis of all sixty-four of the peptides represented by SEQ ID NOs: 4-67 is not always necessary to obtain useful results in the methods described herein. It is possible to employ a sufficient number of peptides selected from among the peptides represented by SEQ ID NOs: 4-67 to provide a statistically reliable result. For example, if the peanut allergy status of a subject is not known, it is generally desirable to analyze a greater number of allergenic epitope-containing peptides selected from among the peptides represented by SEQ ID NOs: 4-67 to ensure that mild to moderate peanut allergy, that may involve reactivity with only a few of the peptides represented by SEQ ID NOs: 4-67, is detectable. Conversely, if a subject is known to have high-intensity peanut allergy, fewer allergenic epitope-containing peptides selected from among the peptides represented by SEQ ID NOs: 4-67 may be sufficient to detect changes in allergy intensity or development of clinical tolerance, because a larger number of the peptides represented by SEQ ID NOs: 4-67 will be initially reactive. However, because changes in allergy intensity and development of clinical tolerance are evidenced by changes in the number of peptides reactive with sera as well as changes in concentration of serum IgE reactive with a particular peptide, it is particularly desirable to include in the assays a large enough set of peptides selected from among the peptides represented by SEQ ID NOs: 4-67 to ensure that changes with respect to a peptide that is diagnostic for a particular subject are not missed. Accordingly, the plurality of allergenic epitope-containing peptides selected from among peptides represented by SEQ ID NOs: 4-67 for use in any of the methods described herein may represent all 64 peptides of SEQ ID NOs: 4-67, a subset of 20-25 peptides, a subset of 15-20 peptides, a subset of 10-15 peptides, a subset of 5-10 peptides, or a subset of 2-5 peptides.

The present disclosure also provides methods for diagnosing a peanut allergy, and/or severity of a peanut allergy, in a subject comprising: contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptides are coupled to a solid support, to form one or more AAI-peptide-solid support complexes; binding an AAI-specific labeling reagent to the AAI-peptide-solid support complex; and detecting binding of the AAI-specific labeling reagent to each AAI-peptide-solid support complex to identify one or more peanut peptides bound to the AAI in the serum or plasma of the subject; wherein recognition of at least one peanut peptide by an AAI in the serum or plasma of the subject indicates that the subject is allergic to peanuts.

In some embodiments, the one or more peanut peptides is derived from ara h 1 allergen (SEQ ID NO:1), ara h 2 allergen (SEQ ID NO:2), and/or ara h 3 allergen (SEQ ID NO:3).

In some embodiments, the one or more peanut peptides is selected from: peptides having at least 3 contiguous amino acids from positions 8 to 66 of ara h 1 allergen; peptides having at least 3 contiguous amino acids from positions 103 to 152 of ara h 1 allergen; peptides having at least 3 contiguous amino acids from positions 176 to 195 of ara h 1 allergen; peptides having at least 3 contiguous amino acids from positions 5 to 40 of ara h 2 allergen; peptides having at least 3 contiguous amino acids from positions 93 to 115 of ara h 3 allergen; peptides having at least 3 contiguous amino acids from positions 30 to 75 of ara h 3 allergen; and/or peptides having at least 3 contiguous amino acids from positions 152 to 167 of ara h 3 allergen.

In some embodiments, the one or more peanut peptides comprise an amino acid sequence selected from any one or more of SEQ ID NOs: 4-67. In some embodiments, the one or more peanut peptides comprise an amino acid sequence selected from any one or more of SEQ ID NOs: 7, 13, 16, 17, 25, 27, 30, 36, 39, 45, 65, and 66. In some embodiments, the one or more peanut peptides comprise an amino acid sequence selected from any one or more of SEQ ID NOs: 39, 45, and 66. In some embodiments, the one or more peanut peptides comprise an amino acid sequence selected from any one or more of SEQ ID NOs: 5, 6, 10, 13, 14, 34, 36, 39, 40, 42, 49, 61, 65, and 66. In some embodiments, the one or more peanut peptides comprise an amino acid sequence selected from any one or more of SEQ ID NOs: 5, 6, 9, 34, 36, 39, and 40. In some embodiments, the one or more peanut peptides comprise an amino acid sequence selected from any one or more of SEQ ID NOs: 29, 39, 42, 44, 45, 51, and 63. In some embodiments, the one or more peanut peptides comprise an amino acid sequence selected from any one or more of SEQ ID NOs: 7, 8, 29, 31, 39, 45, and 61. In some embodiments, the one or more peanut peptides comprise an amino acid sequence selected from any one or more of SEQ ID NOs: 39 and 40. In some embodiments, the one or more peanut peptides comprise an amino acid sequence of SEQ ID NO: 39. In some embodiments, the one or more peanut peptides comprise an amino acid sequence of SEQ ID NO: 40.

In some embodiments, each peanut peptide comprises from about 3 amino acids to about 60 amino acids, from about 4 amino acids to about 60 amino acids, from about 6 amino acids to about 30 amino acids, from about 7 amino acids to about 20 amino acids, from about 10 amino acids to about 16 amino acids, or from about 10 amino acids to about 15 amino acids. In some embodiments, each peanut peptide comprises 15 amino acids.

In some embodiments, the one or more peanut peptides comprise at least 2 peanut peptides, at least 3 peanut peptides, at least 5 peanut peptides, at least 10 peanut peptides, at least 15 peanut peptides, at least 20 peanut peptides, at least 25 peanut peptides, at least 30 peanut peptides, at least 35 peanut peptides, at least 40 peanut peptides, at least 45 peanut peptides, at least 50 peanut peptides, at least 55 peanut peptides, at least 60 peanut peptides, or at least 64 peanut peptides. In some embodiments, the one or more peanut peptides comprises from about 2 to about 64 peanut peptides, from about 2 to about 60 peanut peptides, from about 2 to about 55 peanut peptides, from about 2 to about 50 peanut peptides, from about 2 to about 45 peanut peptides, from about 2 to about 40 peanut peptides, from about 2 to about 35 peanut peptides, from about 2 to about 30 peanut peptides, from about 2 to about 25 peanut peptides, from about 2 to about 20 peanut peptides, from about 2 to about peanut peptides, or from about 2 to about 10 peanut peptides. In some embodiments, the one or more peanut peptides comprises from about 2 to about 64 peanut peptides, from about 5 to about 64 peanut peptides, from about 10 to about 64 peanut peptides, from about 15 to about 64 peanut peptides, from about 20 to about 64 peanut peptides, from about 25 to about 64 peanut peptides, from about 30 to about 64 peanut peptides, from about 35 to about 64 peanut peptides, from about 40 to about 64 peanut peptides, from about 45 to about 64 peanut peptides, from about 50 to about 64 peanut peptides, or from about 55 to about 64 peanut peptides.

In some embodiments, the determination that a subject is allergic to peanuts further takes into account the results of one or more of: total peanut specific IgE (sIgE), peanut component ara h 1 IgE, peanut component ara h 2 IgE, peanut component ara h 3 IgE, total peanut specific IgG4 (sIgG4), peanut component ara h 1 IgG4, peanut component ara h 2 IgG4, peanut component ara h 3 IgG4, skin prick test results, clinical or family history, and/or data from patient or clinician questionnaire. In some embodiments, the determination that a subject is allergic to peanuts further takes into account the results of one or more of: peanut component ara h 1 IgE, peanut component ara h 2 IgE, and/or peanut component ara h 3 IgE.

In some embodiments, the peanut component ara h 1 IgE results comprise results from peanut peptides comprising amino acid sequences chosen from SEQ ID NOs: 7, 13, 16, 17, 25, 27, 30, and 36, or from SEQ ID NOs: 5, 6, 10, 13, 14, 34, and 36, or from SEQ ID NOs: 5, 6, 9, 34, and 36, or from SEQ ID NOs: 7, 8, 29, and 31, or SEQ ID NO:29; the peanut component ara h 2 IgE results comprise results from peanut peptides comprising amino acid sequences chosen from SEQ ID NO:39, or from SEQ ID NOs: 39, 40, 42, and 49, or from SEQ ID NOs: 39 and 40, or from SEQ ID NOs: 39, 42, 44, 45, and 51, or from SEQ ID NOs: 39 and 45, or from SEQ ID NOs: 39 and 40, or from SEQ ID NO: 40; and/or the peanut component ara h 3 IgE results comprise results from peanut peptides comprising amino acid sequences chosen from SEQ ID NO:65, or from SEQ ID NOs: 61, 65, and 66, or from SEQ ID NO: 63, or from SEQ ID NO: 61.

In some embodiments, the determination that a subject is allergic to peanuts further takes into account the results of one or more of: peanut peptide comprising the amino acid sequence of SEQ ID NO:39 IgE, peanut peptide comprising the amino acid sequence of SEQ ID NO:45 IgG4, and peanut peptide comprising the amino acid sequence of SEQ ID NO:66 IgG4; peanut peptide comprising the amino acid sequence of SEQ ID NO:39 IgE, peanut peptide comprising the amino acid sequence of SEQ ID NO:45 IgG4, and skin prick test; peanut peptide comprising the amino acid sequence of SEQ ID NO:39 IgE, peanut peptide comprising the amino acid sequence of SEQ ID NO:45 IgG4, peanut peptide comprising the amino acid sequence of SEQ ID NO:66 IgG4, and sIgE; and peanut peptide comprising the amino acid sequence of SEQ ID NO:39 IgE, peanut peptide comprising the amino acid sequence of SEQ ID NO:45 IgG4, peanut peptide comprising the amino acid sequence of SEQ ID NO:66 IgG4, and peanut component ara h 2.

In some embodiments, the determination that a subject is allergic to peanuts comprises: determining that the subject's sIgE is ≥0.03 kU_{A}/L, which indicates that the subject may be allergic to peanuts, or determining that the subject's sIgE is <0.03 kU_{A}/L, which indicates that the subject is not allergic to peanuts; and when the subject's sIgE is ≥0.03 kU_{A}/L, then determining whether the combination of the peanut peptide comprising the amino acid sequence of SEQ ID NO:39 IgE, peanut peptide comprising the amino acid sequence of SEQ ID NO:45 IgG4, and peanut peptide comprising the amino acid sequence of SEQ ID NO:66 IgG4 is <0.20, which indicates that the subject is not allergic to peanuts, or ≥0.20, which indicates that the subject is allergic to peanuts.

In some embodiments, the Skin Prick Test (SPT) is also taken into account when determining whether a subject is allergic to peanuts. For example, in the single threshold test, if sIgE≤0.10, or SPT≤T1, or h2.008≤0.8, or h2.010≤T2, then the subject is not allergic; otherwise, the subject is allergic. In the double threshold test, if sIgE≤0.10, or SPT≤T1, or h2.008≤0.8, or h2.010≤T2, then the subject is not allergic; otherwise, if h2.010≥T3, then the subject is allergic. T1, T2, and T3 are various thresholds. These thresholds are chosen by the user as part of the algorithm and are assessed based on best performance metrics such as AUC, NPV, and PPV.

In some embodiments, each of the peanut peptides comprises a linker for coupling to the solid support. In some embodiments, the linker is -PEG12-biotin. In some embodiments, the linker can comprise 3, 6, 9, or 12 carbons. In some embodiments, the biotin can be replaced with click chemistry linkers (e.g., azide-DBCO, amine-NHS ester, thiol-malamide, hydrazone, etc.).

In some embodiments, the solid support is a microsphere bead, glass array, silicone array, membrane, or microtiter plate. In some embodiments, each of the solid supports, such as a bead, microtiter plate well, or discrete location on the chromatographic material, is occupied by a single peptide. The solid supports are then contacted with serum or plasma obtained from the subject under conditions appropriate for specific binding of anti-peptide AAI in the serum or plasma (if present) to the peptide on each solid support or discrete location on a solid support to form a peptide-AAI complex on the solid support. Any peptide-AAI complex formed on a solid support is then detected by contacting the complex on each solid support or discrete location on the solid support with a labeling reagent that specifically binds to the complex, typically by binding to the immobilized serum or plasma AAI antibody. A single labeling reagent will generally be used for universal detection of all complexes. The specific peptide-AAI complex may then be identified by its position on the microtiter plate or chromatographic support. When the solid support to which each peptide is conjugated has different spectral properties, the specific peptide-AAI complex may also be identified by analysis of the spectral properties of the solid support associated with the peptide-AAI complex, once the presence of a complex is identified via a detectable signal from the labeling reagent bound to the complex. As an example, the presence or absence of a peptide-AAI complex in each well of a microtiter plate can be determined by binding to the complex an anti-human AAI antibody that is conjugated to a reporter moiety, such as a fluorescent dye, a chromogenic dye, an enzyme label or a radioactive label. Alternatively, the anti-human AAI antibody may be conjugated to a reporter moiety that is not directly detectable, so specific binding of a second, directly detectable reporter moiety to the labeling reagent is necessary for analysis of binding.

In some embodiments, the AAI is IgG, IgM, IgA, and/or IgE. In some embodiments, the IgG is IgG4.

In some embodiments, the AAI-specific labeling reagent is a detectably labeled anti-human IgG4 antibody, detectably labeled anti-human IgM antibody, detectably labeled anti-human IgA antibody, and/or a detectably labeled anti-human IgE antibody. In some embodiments, the detectable label is chosen from phycoerythrin, a fluorescent dye, horse radish peroxidase (HRP), and alkaline phosphatase. In some embodiments, the labeling reagent may be conjugated to a first reporter moiety that is directly detectable, such as a fluorescent dye, radiolabel, or colored dye. In some embodiments, a phycoerythrin (PE) molecule can be directly coupled to an anti-allergy associated immunoglobulin and used for detection. Alternately, the first reporter moiety may be a reporter moiety that is indirectly detectable (e.g., an enzyme label of chromogenic dye) and a specific binding partner for the first reporter moiety can be conjugated to a directly detectable label (the second reporter moiety). For example, a biotin-conjugated anti-AAI antibody can be used in combination with a streptavidin-conjugated fluorescent dye for detection of the biotin-conjugated anti-AAI. In some embodiments, the detectable label can be observed via silver staining, quantum dots, or refraction methodologies.

In some embodiments, the detection of the binding of the AAI-specific labeling reagent to each AAI-peptide-solid support complex is carried out by a multiplex peptide-bead assay for flow cytometric analysis or a lateral flow assay. Any of the foregoing embodiments may be in the form of a microarray immunoassay, wherein each of the plurality of allergenic epitope-containing peptides is bound to a separate well of a microtiter plate and reacted with serum to bind AAI. Bound AAI is detected by binding of an AAI-specific labeling reagent, for example an anti-AAI antibody conjugated to a reporter moiety, such as a fluorescent label. Fluorescence of the bound labeling reagent indicates the presence in the serum or plasma of an antibody to the allergenic epitope contained in the peptide bound to the well. The plurality of allergenic epitope-containing peptides may also be used in a lateral flow immunoassay format, wherein each peptide is immobilized in a discrete area on a porous or chromatographic support, and the serum or plasma is wicked through the support to contact the peptides for binding of AAI to the peptides. In this assay, the AAI-specific labeling reagent may comprise a chromophore or dye conjugated to anti-AAI antibody. The labeling reagent is also wicked through the support to contact the peptide-AAI complexes for binding of the labeling reagent to the complex, which indicates the presence or absence in the serum or plasma of an antibody to the allergenic epitope contained in the peptide immobilized at each discrete location of the support.

Any of the foregoing embodiments may also be in the form of a flow cytometry assay in which each allergenic epitope-containing peptide is conjugated to a separately identifiable solid support suitable for analysis by flow cytometry, such as a bead. Typically, the peptide is conjugated to the solid support by binding to a peptide-specific capture antibody on the solid support or by chemical linkage to the solid support. In some embodiments, the bead with the conjugated allergenic epitope-containing peptide is contacted with the serum or plasma of a subject to bind any peptide-specific AAI that is bound to the bead via the peptide, thus forming a peptide-AAI complex on the bead. An AAI-specific labeling reagent comprising, for example, a fluorescent reporter moiety is then bound to the peptide-AAI complexes and the beads are analyzed quantitatively or qualitatively by flow cytometry. This detects fluorescence from the bound labeling reagent associated with each bead to which an allergenic epitope-containing peptide is conjugated, thereby identifying the peptide and the presence in the serum of AAI that is reactive to it. Presence of AAI reactive to at least one of a plurality of allergenic epitope-containing peptides indicates that the subject is allergic to peanuts, and changes over time in the number of reactive peptides, or changes over time in the concentration of AAI reactive to one or more peptides, indicates an increase in intensity of the allergy, a decrease in the intensity of the allergy, or development of clinical tolerance over that time period.

In some embodiments, the flow cytometry assay may be a multiplex assay, such as the LUMINEX xMAP technology, which uses a microsphere array platform for quantitation and detection of peptides and proteins. Each of the plurality of allergenic epitope-containing peptides is bound to a set of beads with different spectral properties which can be used to identify the associated allergenic epitope-containing peptide by flow cytometry. The sets of beads are then contacted with serum or plasma of a subject to bind peptide-recognizing AAI to each bead to form a peptide-AAI complex on the bead, and an AAI-specific labeling reagent comprising, for example, a fluorescent reporter moiety bound to the AAI of the complex. The beads are analyzed by monitoring the spectral properties of each bead and the amount of associated fluorescence from the bound labeling reagent. This process allows identification of the peptide on the bead, and the presence or absence of serum or plasma AAI that is reactive to it. Results of the assay are interpreted as discussed herein.

A particularly useful quantitative assay for use in any of the methods described herein is a multiplex peptide-bead assay for flow cytometric analysis, such as the LUMINEX exMAP multiplex bead assay, which is a high-throughput alternative to the ELISA. In this assay, polystyrene beads (microspheres) dyed with distinct proportions of red and near-infrared fluorophores are used as the solid support. The peptides may be chemically linked to the beads or bound thereto through peptide-specific capture antibodies coated on the beads. The proportions of the fluorophores define a “spectral address” for each bead population that can be identified by a flow cytometer using digital signal processing. Detection of a third fluorescence color is used for measurement of the fluorescence intensity of the reporter moiety of the labeling reagent bound to the bead. Multiple analytes can be detected simultaneously by binding each peptide to a bead having a specific “spectral address.” Contacting the beads with serum or plasma containing AAI that are specific for the peptide bound to it is followed by addition of anti-human AAI antibodies conjugated to a reporter moiety. In some embodiments, the reporter moiety of the anti-human AAI is biotin and binding to phycoerythyrin (PE)-conjugated streptavidin provides the fluorescent signal for detection. Following binding of the labeling reagent, the beads are analyzed on a dual-laser flow-based detection instrument, such as the LUMINEX 200 or Bio-Rad BIO-PLEX analyzer. One laser classifies the bead and identifies the peptide bound to it. The second laser determines the magnitude of the reporter-derived signal, which is in direct proportion to the amount of bound serum or plasma AAI.

An alternative assay format is a lateral flow or immunochromatographic assay. In such an assay, the selected allergenic epitope containing peptide(s) are immobilized on the porous support and serum or plasma containing the AAI is wicked into contact with the peptide(s) to form immunocomplexes. Further migration of the immunocomplex through the porous support brings it into contact with a specific capture reagent for detection of the immunocomplex using appropriate detection reagents.

In some embodiments, following exposure to peanut allergens, when at least one peptide is moderately or highly reactive with serum or plasma AAI (S/N>2) and reactivity of one or more of the reactive peptides does not decrease at least 2-fold within about six months, the subject is diagnosed as having peanut allergy.

In some embodiments, the methods for diagnosis of peanut allergy are qualitative methods (i.e., based only on presence or absence of AAI reactive to each selected peptide). The presence of AAI moderately or highly reactive with any selected peptide can be considered to indicate some degree of peanut allergy, provided that the reactivity does not substantially diminish within a short period of time such as about six months. The methods may also be semi-quantitative (i.e., the greater the number of peptides reactive with the serum or plasma of the subject, the relatively more intense the allergy and, conversely, the fewer the number of reactive peptides, the relatively less intense the allergy). Serum or plasma reactivity with 5-15 of the peptides may indicate mild to moderate peanut allergy with reactivity within the lower end of this range generally characterized as mild peanut allergy. Serum reactivity with 16-30, 16-25, 16-20, 16-18 or all 64 peptides may indicate moderate to severe peanut allergy, with reactivity within the lower end of this range generally characterized as moderate peanut allergy. In the midrange, serum reactivity with 10-20, 12-18 or 14-16 of the peptides may generally be considered to indicate moderate peanut allergy. It is a particularly useful feature of the peptides that generally no more than about 8-10 are highly reactive (S/N>10) with the serum or plasma of non-allergic individuals and, thus, provide a higher confidence level in the result of the diagnostic assay than conventional assays.

In some embodiments, for analyzing binding to individual peanut peptides, recognition of the peptide by an AAI in the serum or plasma is significant if the value of binding is ≥0.1, ≥0.2, or ≥0.3.

In some embodiments, the methods for diagnosis of peanut allergy are quantitative methods (based on quantitation of the level of AAI reactivity to each selected peptide). In some embodiments, the level of reactivity correlates with the amount of labeling reagent bound to the peptide-AAI complex, with higher levels of signal from the reporter moiety indicating a higher concentration of a particular peptide-specific AAI in the serum or plasma. To obtain the amount or concentration of reporter moiety bound to a particular peptide-AAI complex, the quantity of fluorescence from a fluorescent dye, intensity of color from a colored or chromogenic dye or from an enzyme label, or quantity of radioactivity from a radioactive label is positively correlated with the amount of bound AAI in the complex and therefore its concentration. Methods for measuring these parameters are known in the art. The relative quantities of AAI reactive with any of the peptides can be considered to indicate the degree or intensity of peanut allergy. That is, the higher the level of reactivity of the plurality of selected peptides, or of one or more peptides within the selected peptides, the more intense the allergy. Conversely, the lower the level of reactivity of the plurality of selected peptides, or of one or more peptides within the selected peptides, the less intense the allergy.

The serum or plasma of individuals with mild allergy are reactive with fewer peptides than the serum or plasma of individuals with more intense allergy. The present disclosure, therefore, not only provides methods for diagnosing peanut allergy, it provides methods for determining the intensity of the allergy and methods for determining changes in the intensity of the allergy over time, including detection of development of clinical tolerance to peanuts.

In some embodiments, the number of allergenic epitope-containing peptides that are reactive with the serum or plasma of an allergic subject has a positive correlation with the intensity of the allergic response, i.e., reactivity with fewer peptides indicates a milder allergic response to peanuts and reactivity with more peptides indicates the subject is more highly allergic to peanuts. In some embodiments, the intensity of binding of serum IgE to the peptides (e.g., a measure of IgE concentration in the serum or plasma) correlates with the intensity of the allergic response (i.e., weaker reactivity with all peptides, or with a subset of all the peptides, indicates a more moderate allergic response compared to stronger reactivity with all peptides or with the subset of peptides).

Previously known assays for peanut allergy based on analysis of peptide epitopes in peanut proteins are competitive immunoassays which rely on analysis of the relative affinity of binding of IgE and IgG4 to the epitope. The affinity of antibody binding is believed to be related to whether or not the subject will develop clinical tolerance to peanuts. In contrast, in some embodiments, the methods described herein are partly based on an analysis of the presence or absence of AAI binding to each individual peptide in a set of peanut protein epitopes that correlates with a diagnosis of peanut allergy, with the intensity of the allergic response, and with the potential of a patient to either develop tolerance or experience an increased allergic response based on the number of epitopes (i.e., peptides) bound by IgE in the serum or plasma of the subject. In some embodiments, the methods described herein are partly based on analysis of the concentration of AAIs in the serum or plasma that are reactive with each of the allergenic epitope-containing peptides, which also correlates with the intensity of the allergic response.

As used herein, reference to “non-reactive” or “negative” reactivity with an allergenic epitope-containing peptide means a signal-to-noise ratio (S/N) in the assay that is less than about 2. A typical background signal (N) is that generated by a pool of serum or plasma from non-allergenic individuals. Alternately, negative peptides can be used as the basis for establishing the background signal. As used herein, reference to “weak” or “moderate” reactivity with an allergenic epitope-containing peptide means a S/N of about 2-10, although this value may vary depending on the peptide and the allergy. As used herein, reference to “high” or “strong” reactivity with an allergenic epitope-containing peptide means a S/N of greater than about 10.

Peptides useful in methods for diagnosis of peanut allergy or tolerance thereto, and for detecting increases and decreases in the intensity of the allergy may also include peptides containing non-reactive epitopes of peanut proteins. These peptides are useful as negative controls.

In some embodiments, the analysis of binding of the labeling reagent to each peptide-AAI complex may include analysis of the extent of binding, which indicates a concentration of each peptide-specific AAI in the serum or plasma. A low to moderate serum or plasma reactivity with all of the peptides, or with a subset thereof, indicates a lower concentration of peptide-specific AAI in the serum and mild to moderate peanut allergy, whereas high serum or plasma reactivity with all of the peptides, or a subset thereof, indicates a higher concentration of peptide-specific AAI in the serum and more severe peanut allergy. The analysis of binding for diagnosis of peanut allergy may employ either the number of peptides reactive with serum or plasma, the extent of binding of serum or plasma AAI to the peptides, or both.

The present disclosure also provides methods for detecting development of clinical tolerance to peanuts in a subject that is allergic to peanuts comprising: contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptides are coupled to a solid support, to form one or more AAI-peptide-solid support complexes; binding an AAI-specific labeling reagent to the AAI-peptide-solid support complex; detecting binding of the AAI-specific labeling reagent to each AAI-peptide-solid support complex to identify one or more peanut peptides bound to the AAI in the serum or plasma of the subject; and comparing the identified one or more peanut peptides bound to the AAI in the serum or plasma of the subject, or the concentration of the AAI in the serum or plasma of the subject, with a previously identified panel of one or more peanut peptides bound to the AAI in the serum or plasma of the subject, or a previous concentration of the AAI in the serum or plasma of the subject; wherein development of clinical tolerance to peanuts is indicated when: the subsequent number of peanut peptides recognized by IgE AAI in the serum or plasma of the subject, and/or the subsequent concentration of AAI IgE in the serum or plasma of the subject, is less than the previously identified number of peanut peptides recognized by AAI IgE in the serum or plasma of the subject, and/or less than the previous concentration of AAI IgE in the serum or plasma of the subject; and/or the subsequent number of peanut peptides recognized by IgG4 AAI in the serum or plasma of the subject, and/or the subsequent concentration of AAI IgG4 in the serum or plasma of the subject, is greater than the previously identified number of peanut peptides recognized by AAI IgG4 in the serum or plasma of the subject, and/or greater than the previous concentration of AAI IgG4 in the serum or plasma of the subject.

In some embodiments, the age of a subject undergoing panel identification (e.g., initial, any subsequent, and/or final) can be from about 2 years old to about 50 years old.

In some embodiments, the one or more peanut peptides is as described herein. In some embodiments, the one or more peanut peptides comprises at least two peptides derived from ara h 1 allergen (SEQ ID NO:1), ara h 2 allergen (SEQ ID NO:2), and/or ara h 3 allergen (SEQ ID NO:3).

In some embodiments, the one or more peanut peptides is selected from: peptides having at least 3 contiguous amino acids from positions 8 to 66 of ara h 1 allergen; peptides having at least 3 contiguous amino acids from positions 103 to 152 of ara h 1 allergen; peptides having at least 3 contiguous amino acids from positions 176 to 195 of ara h 1 allergen; peptides having at least 3 contiguous amino acids from positions 5 to 40 of ara h 2 allergen; peptides having at least 3 contiguous amino acids from positions 93 to 115 of ara h 3 allergen; peptides having at least 3 contiguous amino acids from positions 30 to 75 of ara h 3 allergen; and/or peptides having at least 3 contiguous amino acids from positions 152 to 167 of ara h 3 allergen.

In some embodiments, the one or more peanut peptides comprise an amino acid sequence selected from any one or more of SEQ ID NOs: 4-67. In some embodiments, the one or more peanut peptides comprise an amino acid sequence selected from any one or more of SEQ ID NOs: 7, 13, 16, 17, 25, 27, 30, 36, 39, 45, 65, and 66. In some embodiments, the one or more peanut peptides comprise an amino acid sequence selected from any one or more of SEQ ID NOs: 39, 45, and 66. In some embodiments, the one or more peanut peptides comprise an amino acid sequence selected from any one or more of SEQ ID NOs: 5, 6, 10, 13, 14, 34, 36, 39, 40, 42, 49, 61, 65, and 66. In some embodiments, the one or more peanut peptides comprise an amino acid sequence selected from any one or more of SEQ ID NOs: 5, 6, 9, 34, 36, 39, and 40. In some embodiments, the one or more peanut peptides comprise an amino acid sequence selected from any one or more of SEQ ID NOs: 29, 39, 42, 44, 45, 51, and 63. In some embodiments, the one or more peanut peptides comprise an amino acid sequence selected from any one or more of SEQ ID NOs: 7, 8, 29, 31, 39, 45, and 61. In some embodiments, the one or more peanut peptides comprise an amino acid sequence selected from any one or more of SEQ ID NOs: 39 and 40. In some embodiments, the one or more peanut peptides comprise an amino acid sequence of SEQ ID NO: 39. In some embodiments, the one or more peanut peptides comprise an amino acid sequence of SEQ ID NO: 40.

In some embodiments, each peanut peptide comprises from about 3 amino acids to about 60 amino acids, from about 4 amino acids to about 60 amino acids, from about 6 amino acids to about 30 amino acids, from about 7 amino acids to about 20 amino acids, from about 10 amino acids to about 16 amino acids, or from about 10 amino acids to about 15 amino acids. In some embodiments, each peanut peptide comprises 15 amino acids.

In some embodiments, the one or more peanut peptides comprise at least 2 peanut peptides, at least 3 peanut peptides, at least 5 peanut peptides, at least 10 peanut peptides, at least 15 peanut peptides, at least 20 peanut peptides, at least 25 peanut peptides, at least 30 peanut peptides, at least 35 peanut peptides, at least 40 peanut peptides, at least 45 peanut peptides, at least 50 peanut peptides, at least 55 peanut peptides, at least 60 peanut peptides, or at least 64 peanut peptides. In some embodiments, the one or more peanut peptides comprises from about 2 to about 64 peanut peptides, from about 2 to about 60 peanut peptides, from about 2 to about 55 peanut peptides, from about 2 to about 50 peanut peptides, from about 2 to about 45 peanut peptides, from about 2 to about 40 peanut peptides, from about 2 to about 35 peanut peptides, from about 2 to about 30 peanut peptides, from about 2 to about 25 peanut peptides, from about 2 to about 20 peanut peptides, from about 2 to about 15 peanut peptides, or from about 2 to about 10 peanut peptides. In some embodiments, the one or more peanut peptides comprises from about 2 to about 64 peanut peptides, from about 5 to about 64 peanut peptides, from about 10 to about 64 peanut peptides, from about 15 to about 64 peanut peptides, from about 20 to about 64 peanut peptides, from about 25 to about 64 peanut peptides, from about 30 to about 64 peanut peptides, from about 35 to about 64 peanut peptides, from about 40 to about 64 peanut peptides, from about 45 to about 64 peanut peptides, from about 50 to about 64 peanut peptides, or from about 55 to about 64 peanut peptides.

In some embodiments, the determination that a subject is allergic to peanuts further takes into account the results of one or more of: total peanut specific IgE (sIgE), peanut component ara h 1 IgE, peanut component ara h2 IgE, peanut component ara h 3 IgE, skin prick test results, clinical or family history, and/or data from patient or clinician questionnaire.

In some embodiments, each of the peanut peptides comprises a linker for coupling to the solid support. In some embodiments, the linker is -PEG12-biotin. In some embodiments, the linker can comprise 3, 6, 9, or 12 carbons. In some embodiments, the biotin can be replaced with click chemistry linkers (e.g., azide-DBCO, amine-NHS ester, thiol-malamide, hydrazone, etc.). In some embodiments, the solid support is a microsphere bead, glass array, silicone array, membrane, or microtiter plate. In some embodiments, the solid support is as described herein.

In some embodiments, the AAI is IgG, IgM, IgA, and/or IgE. In some embodiments, the IgG is IgG4.

In some embodiments, the AAI-specific labeling reagent is a detectably labeled anti-human IgG4 antibody, detectably labeled anti-human IgM antibody, detectably labeled anti-human IgA antibody, and/or a detectably labeled anti-human IgE antibody. In some embodiments, the detectable label is chosen from phycoerythrin, a fluorescent dye, horse radish peroxidase (HRP), and alkaline phosphatase. In some embodiments, the detectable label can be observed via silver staining, quantum dots, or refraction methodologies. In some embodiments, the detectable label is as described herein.

In some embodiments, the detection of the binding of the AAI-specific labeling reagent to each AAI-peptide-solid support complex is carried out by a multiplex peptide-bead assay for flow cytometric analysis or a lateral flow assay. In some embodiments, the detection assay is as described herein.

The present disclosure also provides methods for detecting an increase in intensity of allergy or adverse event during treatment of allergy to peanuts over time in a subject that is allergic to peanuts comprising: contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptide are coupled to a solid support, to form one or more AAI-peptide-solid support complexes, and wherein the one or more peanut peptides is selected from peptides having at least 3 contiguous amino acids from positions 8 to 66 of ara h 1 allergen, peptides having at least 3 contiguous amino acids from positions 103 to 152 of ara h 1 allergen, peptides having at least 3 contiguous amino acids from positions 176 to 195 of ara h 1 allergen, peptides having at least 3 contiguous amino acids from positions 5 to 40 of ara h 2 allergen, peptides having at least 3 contiguous amino acids from positions 93 to 115 of ara h 3 allergen, peptides having at least 3 contiguous amino acids from positions 30 to 75 of ara h 3 allergen, and/or peptides having at least 3 contiguous amino acids from positions 152 to 167 of ara h 3 allergen; binding an AAI-specific labeling reagent to the AAI-peptide-solid support complex; detecting binding of the AAI-specific labeling reagent to each AAI-peptide-solid support complex to identify one or more peanut peptides bound to the AAI in the serum or plasma of the subject; and comparing the identified one or more peanut peptides bound to the AAI in the serum or plasma of the subject, or the concentration of the AAI in the serum or plasma of the subject, with a previously identified panel of one or more peanut peptides bound to the AAI in the serum of the subject, or a previous concentration of the AAI in the serum or plasma of the subject; wherein an increased intensity of the allergic response to peanuts is indicated when the subsequent number or pattern of reactivity of peanut peptides recognized by AAI in the serum or plasma of the subject, or the subsequent concentration of AAI in the serum or plasma of the subject, is greater than the previously identified number or pattern of reactivity of peanut peptides recognized by AAI in the serum or plasma of the subject, or greater than the previous concentration of AAI in the serum or plasma of the subject.

In some embodiments, the subject may undergo a drift in the identity of allergic peanut peptides from one peanut peptide or one subset of peanut peptides to a different peanut peptide or subset of peanut peptides (a drift to a “hot spot”—see specific subsets of peanut epitopes described herein), which may indicate an increase in the intensity of the peanut allergy or the presence of an adverse event during therapy.

In some embodiments, the one or more peanut peptides is as described herein. In some embodiments, wherein the one or more peanut peptides comprise an amino acid sequence selected from any one or more of SEQ ID NOs: 4-67. In some embodiments, the one or more peanut peptides comprise an amino acid sequence selected from any one or more of SEQ ID NOs: 7, 13, 16, 17, 25, 27, 30, 36, 39, 45, 65, and 66. In some embodiments, the one or more peanut peptides comprise an amino acid sequence selected from any one or more of SEQ ID NOs: 39, 45, and 66. In some embodiments, the one or more peanut peptides comprise an amino acid sequence selected from any one or more of SEQ ID NOs: 5, 6, 10, 13, 14, 34, 36, 39, 40, 42, 49, 61, 65, and 66. In some embodiments, the one or more peanut peptides comprise an amino acid sequence selected from any one or more of SEQ ID NOs: 5, 6, 9, 34, 36, 39, and 40. In some embodiments, the one or more peanut peptides comprise an amino acid sequence selected from any one or more of SEQ ID NOs: 29, 39, 42, 44, 45, 51, and 63. In some embodiments, the one or more peanut peptides comprise an amino acid sequence selected from any one or more of SEQ ID NOs: 7, 8, 29, 31, 39, 45, and 61. In some embodiments, the one or more peanut peptides comprise an amino acid sequence selected from any one or more of SEQ ID NOs: 39 and 40. In some embodiments, the one or more peanut peptides comprise an amino acid sequence of SEQ ID NO: 39. In some embodiments, the one or more peanut peptides comprise an amino acid sequence of SEQ ID NO: 40.

In some embodiments, each peanut peptide comprises from about 3 amino acids to about 60 amino acids, from about 4 amino acids to about 60 amino acids, from about 6 amino acids to about 30 amino acids, from about 7 amino acids to about 20 amino acids, from about 10 amino acids to about 16 amino acids, or from about 10 amino acids to about 15 amino acids. In some embodiments, each peanut peptide comprises 15 amino acids.

In some embodiments, the one or more peanut peptides comprise at least 2 peanut peptides, at least 3 peanut peptides, at least 5 peanut peptides, at least 10 peanut peptides, at least 15 peanut peptides, at least 20 peanut peptides, at least 25 peanut peptides, at least 30 peanut peptides, at least 35 peanut peptides, at least 40 peanut peptides, at least 45 peanut peptides, at least 50 peanut peptides, at least 55 peanut peptides, at least 60 peanut peptides, or at least 64 peanut peptides. In some embodiments, the one or more peanut peptides comprises from about 2 to about 64 peanut peptides, from about 2 to about 60 peanut peptides, from about 2 to about 55 peanut peptides, from about 2 to about 50 peanut peptides, from about 2 to about 45 peanut peptides, from about 2 to about 40 peanut peptides, from about 2 to about 35 peanut peptides, from about 2 to about 30 peanut peptides, from about 2 to about 25 peanut peptides, from about 2 to about 20 peanut peptides, from about 2 to about 15 peanut peptides, or from about 2 to about 10 peanut peptides. In some embodiments, the one or more peanut peptides comprises from about 2 to about 64 peanut peptides, from about 5 to about 64 peanut peptides, from about 10 to about 64 peanut peptides, from about 15 to about 64 peanut peptides, from about 20 to about 64 peanut peptides, from about 25 to about 64 peanut peptides, from about 30 to about 64 peanut peptides, from about 35 to about 64 peanut peptides, from about 40 to about 64 peanut peptides, from about 45 to about 64 peanut peptides, from about 50 to about 64 peanut peptides, or from about 55 to about 64 peanut peptides.

In some embodiments, the determination that a subject is allergic to peanuts further takes into account the results of one or more of: total peanut specific IgE (sIgE), peanut component ara h 1 IgE, peanut component ara h2 IgE, peanut component ara h 3 IgE, skin prick test results, clinical or family history, and/or data from patient or clinician questionnaire.

In some embodiments, each of the peanut peptides comprises a linker for coupling to the solid support. In some embodiments, the linker is -PEG12-biotin. In some embodiments, the linker can comprise 3, 6, 9, or 12 carbons. In some embodiments, the biotin can be replaced with click chemistry linkers (e.g., azide-DBCO, amine-NHS ester, thiol-malamide, hydrazone, etc.). In some embodiments, the solid support is a microsphere bead, glass array, silicone array, membrane, or microtiter plate. In some embodiments, the solid support is as described herein.

In some embodiments, the AAI is IgG, IgM, IgA, and/or IgE. In some embodiments, the IgG is IgG4.

In some embodiments, the AAI-specific labeling reagent is a detectably labeled anti-human IgG4 antibody, detectably labeled anti-human IgM antibody, detectably labeled anti-human IgA antibody, and/or a detectably labeled anti-human IgE antibody. In some embodiments, the detectable label is chosen from phycoerythrin, a fluorescent dye, horse radish peroxidase (HRP), and alkaline phosphatase. In some embodiments, the detectable label can be observed via silver staining, quantum dots, or refraction methodologies. In some embodiments, the detectable label is as described herein.

In some embodiments, the detection of the binding of the AAI-specific labeling reagent to each AAI-peptide-solid support complex is carried out by a multiplex peptide-bead assay for flow cytometric analysis or a lateral flow assay. In some embodiments, the detection assay is as described herein.

Because the degree of binding of each peptide-specific AAI to the peptide-AAI complex on the solid support can be quantitated, the plurality of peptides selected from among peptides represented by SEQ ID NOs: 4-67 are also useful in methods for detecting an increase in the intensity of peanut allergy over time in a subject diagnosed with peanut allergy or development of peanut allergy over time in a subject initially diagnosed as non-allergic. An initial assay is performed on a plurality of peptides selected from among SEQ ID NOs: 4-67 as described herein to provide an initial number of reactive peptides or an initial concentration of each peptide-specific AAI. At a time-point subsequent to the initial assay, the analysis is repeated with the same plurality of peptides selected from among SEQ ID NOs: 4-67 as the initial profile to obtain a subsequent number of reactive peptides or a subsequent concentration of peptide-specific AAI. This method can be summarized as follows: providing an initial profile of a subject's serum or plasma AAI reactivity to a plurality of peptides selected from among SEQ ID NOs: 4-67, wherein the initial profile indicates an initial number of peptides recognized (bound) by AAI in the serum or plasma of the subject or an initial concentration of AAI in the serum or plasma of the subject that recognizes (binds to) each peptide; at a time-point subsequent to the initial profile, contacting each peptide of the same plurality of peptides conjugated to a separately identifiable solid support with serum or plasma from the subject under conditions sufficient to permit binding of AAI in the serum or plasma to the peptide on each solid support, forming a peptide-AAI complex; binding an AAI-specific labeling reagent to the complex, and; analyzing the binding of the labeling reagent to each peptide-AAI complex to identify a subsequent number of peptides recognized by AAI in the serum or plasma of the subject or a subsequent concentration of AAI in the serum or plasma of the subject that reacts with each selected peptide.

The methods for detecting an increase in intensity of the allergy may make use of any appropriate assay format, including those described herein. Examples of the types of analyses available for analyzing binding of the labeling reagent are also as described herein. An increase in the number of peptides reactive with AAI at the subsequent time-point compared to the initial profile (including an increase compared to no peptides reactive with AAI in the initial profile), or an increase in intensity of binding of AAI to any of the peptides at the subsequent time-point compared to the initial profile (including an increase from no binding to a particular peptide in the initial profile to detectable binding at the subsequent time-point), indicates an increase in the intensity of peanut allergy in a subject previously diagnosed with peanut allergy or development of peanut allergy in the previously non-allergic subject. As discussed herein, comparing the initial profile of a subject to that of a subsequent time point may be used to predict the subject's increase in severity or lower tolerance in a particular allergy, or to predict the likelihood of development of clinical or natural tolerance to the allergen.

The present disclosure also provides methods of sensitizing an infant to one or more peanut allergens to induce tolerance or non-allergy to peanuts comprising administering one or more peanut peptides to the infant, wherein the one or more peanut peptides are derived from ara h 1 allergen (SEQ ID NO:1), ara h 2 allergen (SEQ ID NO:2), and/or ara h 3 allergen (SEQ ID NO:3).

In some embodiments, the age of a subject undergoing administration (e.g., initial administration, any subsequent administration, and/or last administration) can be less than about one year old, less than about 2 years old, less than about 3 years old, less than about 4 years old, less than about 5 years old, or less than about 6 years old. The amount of total peptide or individual peptide can be about 1 gram or less per dose. The administration can be oral, sublingual, intradermal, subcutaneous, inhalation, or epicutaneous.

In some embodiments, the one or more peanut peptides is as described herein. In some embodiments, the one or more peanut peptides is selected from: peptides having at least 3 contiguous amino acids from positions 8 to 66 of ara h 1 allergen; peptides having at least 3 contiguous amino acids from positions 103 to 152 of ara h 1 allergen; peptides having at least 3 contiguous amino acids from positions 176 to 195 of ara h 1 allergen; peptides having at least 3 contiguous amino acids from positions 5 to 40 of ara h 2 allergen; peptides having at least 3 contiguous amino acids from positions 93 to 115 of ara h 3 allergen; peptides having at least 3 contiguous amino acids from positions 30 to 75 of ara h 3 allergen; and/or peptides having at least 3 contiguous amino acids from positions 152 to 167 of ara h 3 allergen.

In some embodiments, the one or more peanut peptides comprise an amino acid sequence selected from any one or more of SEQ ID NOs: 4-67. In some embodiments, the one or more peanut peptides comprise an amino acid sequence selected from any one or more of SEQ ID NOs: 7, 13, 16, 17, 25, 27, 30, 36, 39, 45, 65, and 66. In some embodiments, the one or more peanut peptides comprise an amino acid sequence selected from any one or more of SEQ ID NOs: 39, 45, and 66. In some embodiments, the one or more peanut peptides comprise an amino acid sequence selected from any one or more of SEQ ID NOs: 5, 6, 10, 13, 14, 34, 36, 39, 40, 42, 49, 61, 65, and 66. In some embodiments, the one or more peanut peptides comprise an amino acid sequence selected from any one or more of SEQ ID NOs: 5, 6, 9, 34, 36, 39, and 40. In some embodiments, the one or more peanut peptides comprise an amino acid sequence selected from any one or more of SEQ ID NOs: 29, 39, 42, 44, 45, 51, and 63. In some embodiments, the one or more peanut peptides comprise an amino acid sequence selected from any one or more of SEQ ID NOs: 7, 8, 29, 31, 39, 45, and 61. In some embodiments, the one or more peanut peptides comprise an amino acid sequence selected from any one or more of SEQ ID NOs: 39 and 40. In some embodiments, the one or more peanut peptides comprise an amino acid sequence of SEQ ID NO: 39. In some embodiments, the one or more peanut peptides comprise an amino acid sequence of SEQ ID NO: 40.

In some embodiments, each peanut peptide comprises from about 3 amino acids to about 60 amino acids, from about 4 amino acids to about 60 amino acids, from about 6 amino acids to about 30 amino acids, from about 7 amino acids to about 20 amino acids, from about amino acids to about 16 amino acids, or from about 10 amino acids to about 15 amino acids. In some embodiments, each peanut peptide comprises 15 amino acids.

In some embodiments, the one or more peanut peptides comprise at least 2 peanut peptides, at least 3 peanut peptides, at least 5 peanut peptides, at least 10 peanut peptides, at least 15 peanut peptides, at least 20 peanut peptides, at least 25 peanut peptides, at least 30 peanut peptides, at least 35 peanut peptides, at least 40 peanut peptides, at least 45 peanut peptides, at least 50 peanut peptides, at least 55 peanut peptides, at least 60 peanut peptides, or at least 64 peanut peptides. In some embodiments, the one or more peanut peptides comprises from about 2 to about 64 peanut peptides, from about 2 to about 60 peanut peptides, from about 2 to about 55 peanut peptides, from about 2 to about 50 peanut peptides, from about 2 to about 45 peanut peptides, from about 2 to about 40 peanut peptides, from about 2 to about 35 peanut peptides, from about 2 to about 30 peanut peptides, from about 2 15 to about 25 peanut peptides, from about 2 to about 20 peanut peptides, from about 2 to about peanut peptides, or from about 2 to about 10 peanut peptides. In some embodiments, the one or more peanut peptides comprises from about 2 to about 64 peanut peptides, from about 5 to about 64 peanut peptides, from about 10 to about 64 peanut peptides, from about 15 to about 64 peanut peptides, from about 20 to about 64 peanut peptides, from about 25 to about 64 peanut peptides, from about 30 to about 64 peanut peptides, from about 35 to about 64 peanut peptides, from about 40 to about 64 peanut peptides, from about 45 to about 64 peanut peptides, from about 50 to about 64 peanut peptides, or from about 55 to about 64 peanut peptides.

In some embodiments, the initial detection of development of clinical tolerance can be used to predict if a patient will either develop a natural tolerance to the allergy or be responsive to therapy. In some embodiments, an allergic subject is exposed to the immunogen (immunotherapy) prior to analyzing the initial profile. If at the subsequent time-point there is a reduction of at least 2-fold in serum concentration of all AAIs that were highly reactive with peptides in the initial profile, it is likely that the subject will develop either clinical or natural tolerance to peanuts. If at the subsequent time-point there is a reduction of at least 2-fold in serum concentration of fewer than all AAIs that were highly reactive with peptides in the initial profile, the subject is likely to develop only partial clinical or natural tolerance to peanuts.

The plurality of peptides selected from among peptides represented by SEQ ID NOs: 4-67 are also useful in methods for detecting development of clinical tolerance to peanut proteins in a subject diagnosed with peanut allergy. In some embodiments, the assay generally is as described herein for detection of an increase in allergy intensity, is performed first at an initial time-point to establish an initial profile of serum or plasma AAI reactivity with the plurality of peptides selected from among SEQ ID NOs: 4-67. The initial profile is based on semi-quantitative or quantitative analysis of serum or plasma reactivity with the selected peptides, as discussed herein. The selected peptides conjugated to the solid supports are then contacted with serum or plasma from the subject obtained at a time-point subsequent to the initial profile and the assay is conducted as described herein with semi-quantitation or quantitation of the intensity of peanut allergy at the subsequent time-point. A reduction in the number of peptides reactive with AAI at the subsequent time-point as compared to the initial profile, or a reduction in intensity of binding of AAI to any of the peptides at the subsequent time-point as compared to the initial profile, particularly at least a 2-fold reduction, indicates development of clinical tolerance to peanut proteins. It will be appreciated that development of clinical tolerance to peanut proteins in a subject previously diagnosed with peanut allergy also indicates a decrease in allergy intensity over the time period between the initial profile and the subsequent time-point, and that the method can also be used to detect and predict such decreases in allergy intensity over time.

Immunotherapy approaches to treat allergy have largely focused on using whole protein or peanut extracts to treat and desensitize patients. Peptides are an attractive alternative that may represent a more focused and safer approach in the treatment of peanut allergy. Particular peptides from the important IgE reactive regions on Ara h1, Ara h 2, and ara h 3 (e.g., Ara h 1: 8-66, Ara h 2: 5-40, and Ara h 3 93-115) may have certain utility for the treatment of peanut allergy either individually, in combination, or in combination with other therapeutic approaches. Particular peptides can be administered via an oral, sublingual, intradermal, sub-cutaneous, inhaled, or epicutaneous route to treat allergy.

The present disclosure also provides kits comprising: one or more allergenic epitope-containing peanut peptides derived from ara h 1 allergen (SEQ ID NO:1), ara h 2 allergen (SEQ ID NO:2), and/or ara h 3 allergen (SEQ ID NO:3), wherein each peanut peptide is coupled to a solid support; and an allergy associated immunoglobulin (AAI)-specific labeling reagent; packaged together and including instructions for use.

In some embodiments, the kits further comprise one or more of a binding buffer, a wash buffer, and a detection buffer. In some embodiments, the kits further comprise a reporter moiety that specifically binds to the AAI-specific labeling reagent.

In some embodiments, the one or more peanut peptides in the kits is selected from: peptides having at least 3 contiguous amino acids from positions 8 to 66 of ara h 1 allergen; peptides having at least 3 contiguous amino acids from positions 103 to 152 of ara h 1 allergen; peptides having at least 3 contiguous amino acids from positions 176 to 195 of ara h 1 allergen; peptides having at least 3 contiguous amino acids from positions 5 to 40 of ara h 2 allergen; peptides having at least 3 contiguous amino acids from positions 93 to 115 of ara h 3 allergen; peptides having at least 3 contiguous amino acids from positions 30 to 75 of ara h 3 allergen; and/or peptides having at least 3 contiguous amino acids from positions 152 to 167 of ara h 3 allergen.

In some embodiments, the one or more peanut peptides in the kits are as described herein. In some embodiments, the one or more peanut peptides in the kits comprise an amino acid sequence selected from any one or more of SEQ ID NOs: 4-67. In some embodiments, the one or more peanut peptides comprise an amino acid sequence selected from any one or more of SEQ ID NOs: 7, 13, 16, 17, 25, 27, 30, 36, 39, 45, 65, and 66. In some embodiments, the one or more peanut peptides comprise an amino acid sequence selected from any one or more of SEQ ID NOs: 39, 45, and 66. In some embodiments, the one or more peanut peptides comprise an amino acid sequence selected from any one or more of SEQ ID NOs: 5, 6, 10, 13, 14, 34, 36, 39, 40, 42, 49, 61, 65, and 66. In some embodiments, the one or more peanut peptides comprise an amino acid sequence selected from any one or more of SEQ ID NOs: 5, 6, 9, 34, 36, 39, and 40. In some embodiments, the one or more peanut peptides comprise an amino acid sequence selected from any one or more of SEQ ID NOs: 29, 39, 42, 44, 45, 51, and 63. In some embodiments, the one or more peanut peptides comprise an amino acid sequence selected from any one or more of SEQ ID NOs: 7, 8, 29, 31, 39, 45, and 61. In some embodiments, the one or more peanut peptides comprise an amino acid sequence selected from any one or more of SEQ ID NOs: 39 and 40. In some embodiments, the one or more peanut peptides comprise an amino acid sequence of SEQ ID NO: 39. In some embodiments, the one or more peanut peptides comprise an amino acid sequence of SEQ ID NO: 40.

In some embodiments, each peanut peptide in the kits comprises from about 3 amino acids to about 60 amino acids, from about 4 amino acids to about 60 amino acids, from about 6 amino acids to about 30 amino acids, from about 7 amino acids to about 20 amino acids, from about 10 amino acids to about 16 amino acids, or from about 10 amino acids to about 15 amino acids. In some embodiments, each peanut peptide comprises 15 amino acids.

In some embodiments, the one or more peanut peptides in the kits comprise at least 2 peanut peptides, at least 3 peanut peptides, at least 5 peanut peptides, at least 10 peanut peptides, at least 15 peanut peptides, at least 20 peanut peptides, at least 25 peanut peptides, at least 30 peanut peptides, at least 35 peanut peptides, at least 40 peanut peptides, at least 45 peanut peptides, at least 50 peanut peptides, at least 55 peanut peptides, at least 60 peanut peptides, or at least 64 peanut peptides. In some embodiments, the one or more peanut peptides comprises from about 2 to about 64 peanut peptides, from about 2 to about 60 peanut peptides, from about 2 to about 55 peanut peptides, from about 2 to about 50 peanut peptides, from about 2 to about 45 peanut peptides, from about 2 to about 40 peanut peptides, from about 2 to about 35 peanut peptides, from about 2 to about 30 peanut peptides, from about 2 to about 25 peanut peptides, from about 2 to about 20 peanut peptides, from about 2 to about 15 peanut peptides, or from about 2 to about 10 peanut peptides. In some embodiments, the one or more peanut peptides comprises from about 2 to about 64 peanut peptides, from about 5 to about 64 peanut peptides, from about 10 to about 64 peanut peptides, from about 15 to about 64 peanut peptides, from about 20 to about 64 peanut peptides, from about 25 to about 64 peanut peptides, from about 30 to about 64 peanut peptides, from about 35 to about 64 peanut peptides, from about 40 to about 64 peanut peptides, from about 45 to about 64 peanut peptides, from about 50 to about 64 peanut peptides, or from about 55 to about 64 peanut peptides.

In some embodiments, each of the peanut peptides in the kits comprises a linker for coupling to the solid support. In some embodiments, the linker is -PEG12-biotin. In some embodiments, the linker can comprise 3, 6, 9, or 12 carbons. In some embodiments, the biotin can be replaced with click chemistry linkers (e.g., azide-DBCO, amine-NHS ester, thiol-malamide, hydrazone, etc.). In some embodiments, the solid support is a microsphere bead, glass array, silicone array, membrane, or microtiter plate. In some embodiments, the solid support is as described herein.

In some embodiments, the AAI-specific labeling reagent is a detectably labeled anti-human IgG4 antibody, detectably labeled anti-human IgM antibody, detectably labeled anti-human IgA antibody, and/or a detectably labeled anti-human IgE antibody. In some embodiments, the detectable label is chosen from phycoerythrin, a fluorescent dye, horse radish peroxidase (HRP), and alkaline phosphatase. In some embodiments, the detectable label can be observed via silver staining, quantum dots, or refraction methodologies. In some embodiments, the labeling reagent is as described herein.

For the convenience of the user, the reagents for use in any of the methods described herein may be packaged together in the form of a kit comprising a plurality of allergenic epitope-containing peptides selected from among the peptides represented by SEQ ID NOs: 4-67 or any of the useful subgroups, a labeling reagent comprising an anti-human IgE antibody conjugated to a first reporter moiety and, optionally (if required for indirect detection) a second reporter moiety that specifically binds to the labeling reagent. The kit will typically include instructions for use of these reagents in one or more of the methods described herein.

In some embodiments, the kit may comprise an anti-human AAI antibody that may be provided conjugated to a reporter moiety that can be directly detected. Directly detectable reporter moieties are those that can be identified and/or quantitated without the need for binding to a specific binding partner. Examples of directly-detectable reporter moieties that may be conjugated to the anti-human AAI antibody include fluorescent dyes, colored dyes, chromogenic dyes, and enzyme labels that can be detected by a subsequent chemical reaction, and radiolabels. In some embodiments, the anti-human AAI antibody may be provided conjugated to a reporter moiety that is indirectly detectable, i.e., a reporter moiety that is not itself detectable but which undergoes a reaction or interaction with a second reporter moiety that comprises a directly detectable reporter moiety, such as a specific binding partner for the reporter moiety conjugated to a directly detectable label. Examples of indirectly-detectable reporter moieties include biotin, digoxigenin, and other haptens that are detectable upon subsequent binding of a secondary antibody (e.g., anti-digoxigenin) or other binding partner (e.g., streptavidin) which is labeled for direct detection. It will be understood that any of these labeling reagents and reporter moieties are useful in the appropriate assay format in the methods described herein and as components of the kits. In some embodiments, in a kit for performing the flow cytometry multiplex assay described herein, the components of the kit may comprise a plurality of allergenic epitope-containing peptides selected from among the peptides represented by SEQ ID NOs: 4-67, a biotinylated anti-human AAI antibody (labeling reagent with first reporter moiety), and streptavidin conjugated to PE (second reporter moiety).

The plurality of allergenic epitope-containing peptides selected from among SEQ ID NOs: 4-67 for inclusion in any of the foregoing kits may represent all 64 peptides of SEQ ID NOs: 4-67, a subset of 20-25 peptides, a subset of 15-20 peptides, a subset of 10-15 peptides, a subset of 5-10 peptides or a subset of 2-5 peptides. The plurality of allergenic epitope-containing peptides selected from among SEQ ID NOs: 4-67 for inclusion in any of the foregoing kits may also represent one or more of the related peptides subgroups.

The methods described herein can be used as: 1) screening assays (e.g., high-risk patient due to family history of a peanut allergy to inform therapeutic approach/intervention (patient may be exposed to allergen or avoid due to potential severe reaction); patients demonstrating allergy or sensitivity to peanuts to guide patients diet and/or likelihood to outgrow (initiation of desensitization the therapy via AIT)); 2) diagnostic assays (e.g., for the diagnosis of a patient with a suspected peanut allergy; to stratify patients based on severity of their allergic response; based on exposure level; based on presentation of the allergen (based on level of allergen protein denaturation); confirm that the reactivity is due to a specific protein within peanuts and not a cross reactivity response (report reactivity based on component); 3) predictor assays (e.g., predict therapeutic outcome; determine if a patient will respond to therapy; predict optimal therapeutic approach; predict effective starting dose and/or length of therapy (e.g., 1 year vs. 3 years); 4) prognostic assays (e.g., determine if a patient will outgrow their allergy; determine whether allergy will become more severe over time independent of therapy; help characterize the disease to influence treatment decision and guide patient and drug/dose selection); 4) monitoring assays (e.g., adverse event as a result of AIT; and determination of a particular outcome (e.g., desensitization, sustained unresponsiveness, tolerance level, and regression)).

The peanut peptide sequences include: ATHAKSSPYQKKTEN (ara h 1.008; SEQ ID NO:4), LQSCQQEPDDLKQKA (ara h 1.015; SEQ ID NO:5), RCTKLEYDPRCVYDP (ara h 1.021; SEQ ID NO:6), KLEYDPRCVYDPRGH (ara h 1.022; SEQ ID NO:7), YDPRG HTGTTNQRSP (ara h 1.025; SEQ ID NO:8), RSPPGERTRGRQPGD (ara h 1.029; SEQ ID NO:9), PGERTRGRQPGDYDD (ara h 1.030; SEQ ID NO:10), PGDYDDDRRQPRREE (ara h 1.033; SEQ ID NO:11), DRRQPRREEGGRWGP (ara h 1.035; SEQ ID NO:12), AGP REREREEDWRQP (ara h 1.040; SEQ ID NO:13), REREREEDWRQPRED (ara h 1.041; SEQ ID NO:14), RQPREDWRRPSHQQP (ara h 1.044; SEQ ID NO:15), REDWRRPSHQQ PRKI (ara h 1.045; SEQ ID NO:16), PSHQQPRKIRPEGRE (ara h 1.047; SEQ ID NO:17), RPEGREGEQEWGTPG (ara h 1.050; SEQ ID NO:18), REETSRNNPFYFPSR (ara h 1.056; SEQ ID NO:19), NNPFYFPSRRFSTRY (ara h 1.058; SEQ ID NO:20), SGFISYILNRHDN QN (ara h 1.090; SEQ ID NO:21), SMPVNTPGQFEDFFP (ara h 1.097; SEQ ID NO:22), RDQSSYLQGFSRNTL (ara h 1.103; SEQ ID NO:23), SEEEGDITNPINLRE (ara h 1.130; SEQ ID NO:24), EGDITNPINLREGEP (ara h 1.131; SEQ ID NO:25), NNFGKLFEVKPDK KN (ara h 1.137; SEQ ID NO:26), RYTARLKEGDVFIMP (ara h 1.167; SEQ ID NO:27), DVFIMPAAHPVAINA (ara h 1.170; SEQ ID NO:28), PVAINASSELHLLGF (ara h 1.173; SEQ ID NO:29), LHLLGFGINAENNHR (ara h 1.176; SEQ ID NO:30), AENNHRIFLAGD KDN (ara h 1.179; SEQ ID NO:31), NHRIFLAGDKDNVID (ara h 1.180; SEQ ID NO:32), VIDQIEKQAKDLAFP (ara h 1.184; SEQ ID NO:33), KQAKDLAFPGSGEQV (ara h 1.186; SEQ ID NO:34), KDLAFPGSGEQVEKL (ara h 1.187; SEQ ID NO:35), SHFVSARPQSQS QSP (ara h 1.194; SEQ ID NO:36), QEEENQGGKGPLLSI (ara h 1.203; SEQ ID NO:37), AAHASARQQWELQGD (ara h 2.005; SEQ ID NO:38), WELQGDRRCQSQLER (ara h 2.008; SEQ ID NO:39), RRCQSQLERANLRPC (ara h 2.010; SEQ ID NO:40), RPCEQH LMQKIQRDE (ara h 2.014; SEQ ID NO:41), KIQRDEDSYERDPYS (ara h 2.017; SEQ ID NO:42), RDEDSYERDPYSPSQ (ara h 2.018; SEQ ID NO:43), DSYERDPYSPSQDPY (ara h 2.019; SEQ ID NO:44), PYSPSQDPYSPSPYD (ara h 2.021; SEQ ID NO:45), CCNELNE FENNQRCM (ara h 2.030; SEQ ID NO:46), ELNEFENNQRCMCEA (ara h 2.031; SEQ ID NO:47), LQQIMENQSDRLQGR (ara h 2.036; SEQ ID NO:48), IMENQSDRLQGRQQE (ara h 2.037; SEQ ID NO:49), NQSDRLQGRQQEQQF (ara h 2.038; SEQ ID NO:50), QGR QQEQQFKRELRN (ara h 2.040; SEQ ID NO:51), KRELRNLPQQCGLRA (ara h 2.043; SEQ ID NO:52), LPQQCGLRAPQRCDL (ara h 2.045; SEQ ID NO:53), LRRNALRRPFY SNAP (ara h 3.018; SEQ ID NO:54), HYEEPHTQGRRSQSQ (ara h 3.030; SEQ ID NO:55), EPHTQGRRSQSQRPP (ara h 3.031; SEQ ID NO:56), QGEDQSQQQRDSHQK (ara h 3.037; SEQ ID NO:57), NTEQEFLRYQQQSRQ (ara h 3.060; SEQ ID NO:58), PYSPQSQP RQEEREF (ara h 3.068; SEQ ID NO:59), EGGNIFSGFTPEFLE (ara h 3.079; SEQ ID NO:60), NIFSGFTPEFLEQAF (ara h 3.080; SEQ ID NO:61), AIVTVRGGLRILSPD (ara h 3.092; SEQ ID NO:62), TVRGGLRILSPDRKR (ara h 3.093; SEQ ID NO:63), EYDEDEYE YDEEDRR (ara h 3.100; SEQ ID NO:64), YEYDEEDRRRGRGSR (ara h 3.102; SEQ ID NO:65), IANLAGENSVIDNLP (ara h 3.152; SEQ ID NO:66), and RQLKNNNPFKFFVPP (ara h 3.162; SEQ ID NO:67).

Any one or more of these peptides can be conjugated to, for example, a -PEG12-Biotin at its carboxy terminus.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: Analysis of LEAP Cohort Using Epitope Assay

As background, a randomized controlled trial (RCT) (e.g., Learning Early About Peanut allergy (LEAP)) was previously carried out to determine the best strategy for preventing peanut allergy in young children. The LEAP trial consisted of 640 children between 4 and 11 months of age who have been identified as having a high risk of peanut allergy. The children were divided into two groups: avoidance and consumption (a peanut containing snack with greater than 3 meals; 6 g of peanut protein per week). The results of this trial indicated that the proportion of the children that developed peanut allergy by 5 years of age was from 4-fold to 6-fold greater in the avoidance group compared to the consumption group as determined by an oral food challenge (OFC). The results of the LEAP trial are disclosed in, for example, Toit et al., N. Engl. J. Med., 2015, 372, 803-813. Thus trial led to a change in the Guidelines for the Prevention of Peanut Allergy in the United States.

### Methods:

To assess the importance of individual epitopes, epitope combinations, and epitopes combined with other clinical or diagnostics methods (e.g., Skin Prick Test, peanut-specific IgE, patient history, and peanut component IgE), a subset of the LEAP patient cohort was further evaluated using the epitope test described herein. In particular, a subset of 341 LEAP Per Protocol subjects who had at least 2 additional aliquots of plasma from time points during the LEAP trial was selected. The diagnostic classification (i.e., allergy status at 5 years—Visit 60) of the 341 subjects is shown in Table 1.

TABLE 1

| | Avoiders | Consumers |
|---|---|---|
| Outcome n (%) | n = 172 | N = 169 |
| Allergic | 38 (22.1%) | 0 (0.0%) |
| Sensitized | 84 (48.8%) | 119 (70.4%) |
| Not Allergic | 50 (29.1%) | 50 (29.6%) |

The diagnostic classification was determined as follows. “Allergic” subjects exhibited clinical allergic symptoms after a peanut OFC at Visit 60. “Sensitized” subjects were IgE sensitized, but not allergic, had at least one peanut-specific IgE>0.1 kU_{A}/L for the first three visits, and passed an OFC at Visit 60. “Not Allergic” subjects (100 subjects randomly selected in a 1:1 ratio from Avoiders and Consumers) were not sensitized and not allergic, were negative for a Skin Prick Test (SPT) and had a peanut-specific IgE≤0.1 kU/L* for the first three visits, and passed an OFC at Visit 60.

The assay to assess subject IgE and IgG4 reactivity at 64 specific epitopes, beginning with epitope selection, bead coupling, sample assaying, data acquisition, and statistical analysis, was carried out as depicted as follows. Briefly, chemically modified, synthetic, peanut peptides (15 amino acids in length; representing 50 linear epitopes from three major peanut proteins—Ara h1-27 epitopes, Ara h2-13 epitopes, and Ara h3-10 epitopes,) containing a C-terminal biotin/PEG12 linker were coupled to Luminex LumAvidin microspheres at a concentration of 4,000 nM for 30 minutes at room temperature with constant, gentle rotation protected from light in a buffer of PBS/1% BSA. Prior to coupling, the stock microspheres were centrifuged for 2 minutes at 10,000×g, sonicated in a water bath sonicator, and vortexed at medium speed to resuspend the microspheres. Multiple vials of prepared microspheres were subsequently combined and pelleted again before removal of the supernatant and resuspension in PBS/1% BSA for coupling. Coupled microspheres were washed twice by centrifugation with PBS-TBN/azide buffer (PBS/0.1% BSA/0.02% Tween-20/0.05% sodium azide), resuspended in PBS-TBN, and counted on a glass hemocytometer.

Sixty four different peanut peptide coupled bead regions were combined together to form a “64-plex” at a concentration of 1,000 beads/plex and a Luminex assay performed. Triplicate wells of human plasma samples were diluted 1:10 in PBS-TBN (PBS/0.1% BSA/0.02% Tween-20) buffer and incubated with the peptide coupled microsphere plex for 2 hours at room temperature with shaking protected from light. Plates were subsequently washed twice with PBS-TBN and secondary detection antibodies consisting of mouse anti-human IgE or IgG4 directly coupled to phycoerythrin added to the wells and incubated at room temperature for 30 minutes with shaking protected from light. Plates were washed three times with PBS-TBN, resuspended in 100 μL of PBS-TBN, and transferred to a secondary plate prior to reading on a Luminex-100 set for high RP1 and a minimum count of 50 beads per region. Each plate contained an inter-plate control sample (IPC) comprising a mixture of plasma from 150 randomly selected patient samples run in triplicate as well as a buffer only (negative) control run in triplicate. Raw data obtained from the epitope assay was reported as Median Fluorescence Intensities (MFI). All data was log 2 transformed using the formula: epitope value=log 2(MFI+0.05)−average(log 2(Buffer+0.05)).

### Assessment of Epitope Changes Over Time: Comparison of Avoiders Verses Consumers:

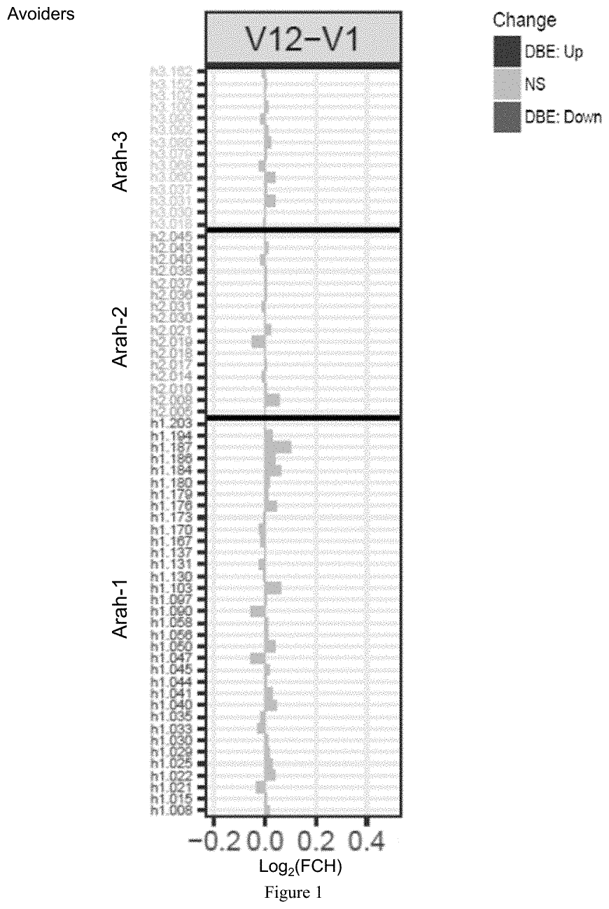

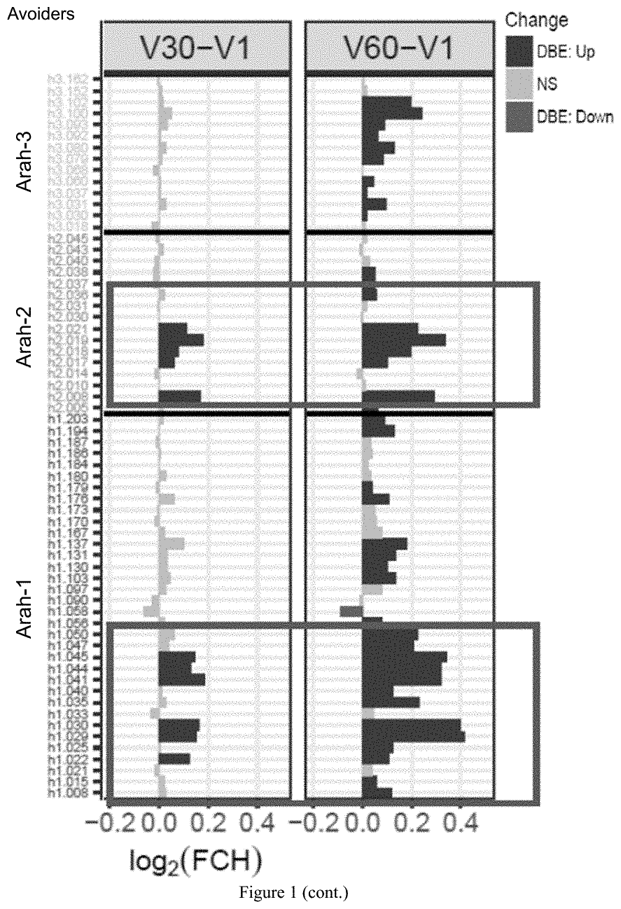

To assess IgE epitope differences between study groups (Avoiders and Consumers), the fold change in each epitope value across study visits compared to the baseline visit at the beginning of the study (V12-V1, V30-V1, and V60-V1) was determined. The results are plotted by epitope and summarized in FIG. 1. Nineteen epitopes with greater change in Avoiders than Consumers was observed. The results indicate that IgE sequential epitope-specific antibodies developed early in children in the Avoider group who developed peanut allergy by 5 years old (visit 60). Sequential epitope-specific IgE antibodies developed predominantly in two regions: Ara h 1: 008-066 and Ara h 2: 005-040. In addition, there appears to be a late response to Ara h 3: 93-115. Late changes in IgE-specific binding are mainly in the peanut Avoider group; they are specific to two regions and they persist increasing the binding over time. A small increase of IgE early (V12) in the Consumers was observed, but not to the same epitopes as seen in subjects developing allergy. In sum, IgE was observed to develop early in the Consumer group but not at the regions overlapping with patients who developed allergy. These data suggest that an IgE response to certain peanut epitopes may be both natural and transient. Taken together, the data suggest that certain epitopes in these peanut specific proteins may be indicative of an IgE allergy response.

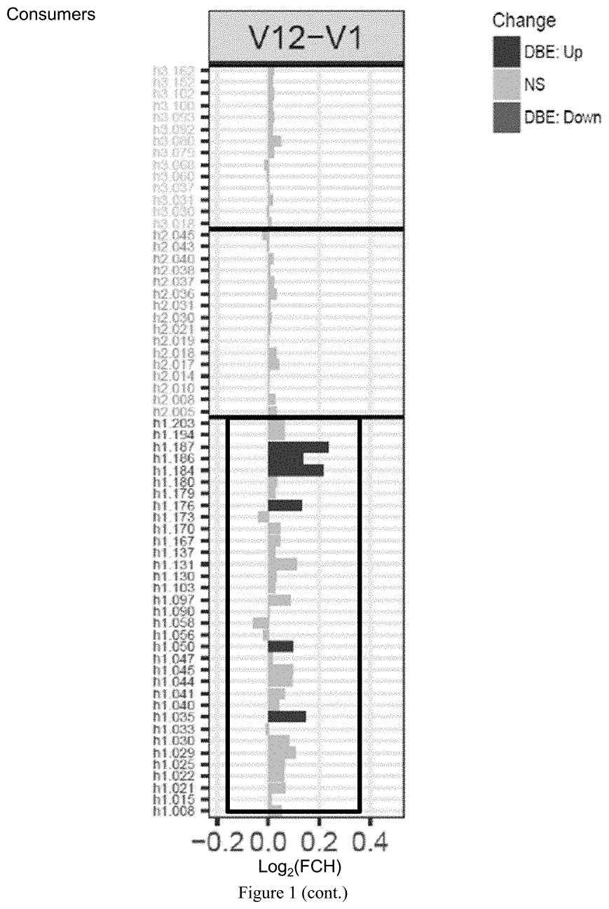

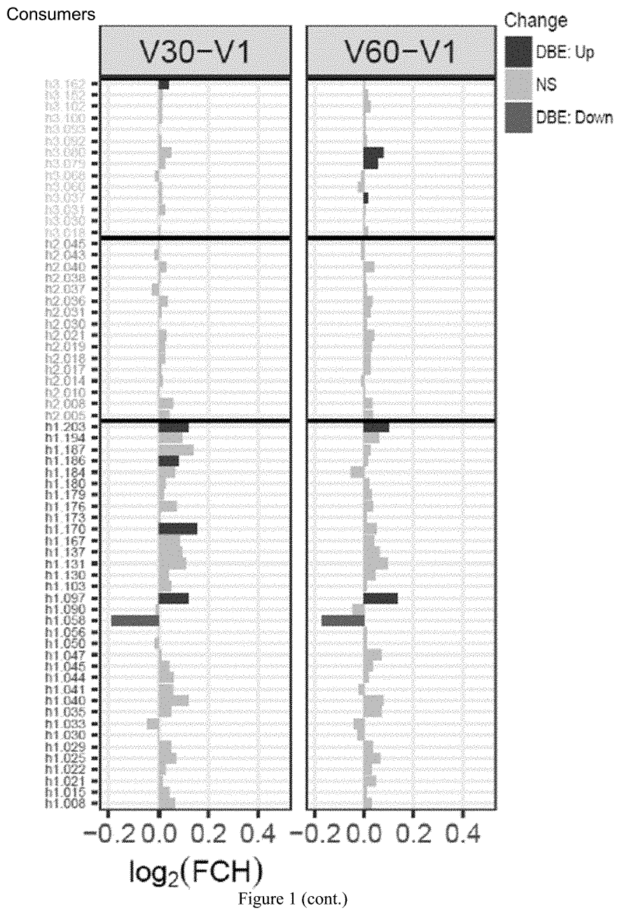

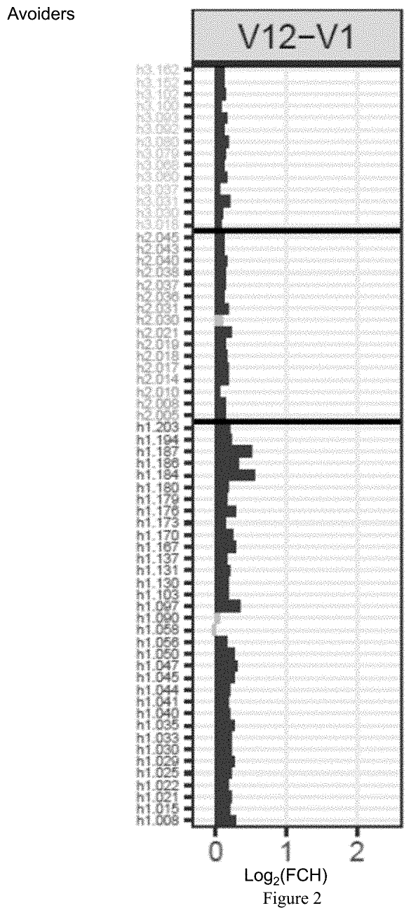

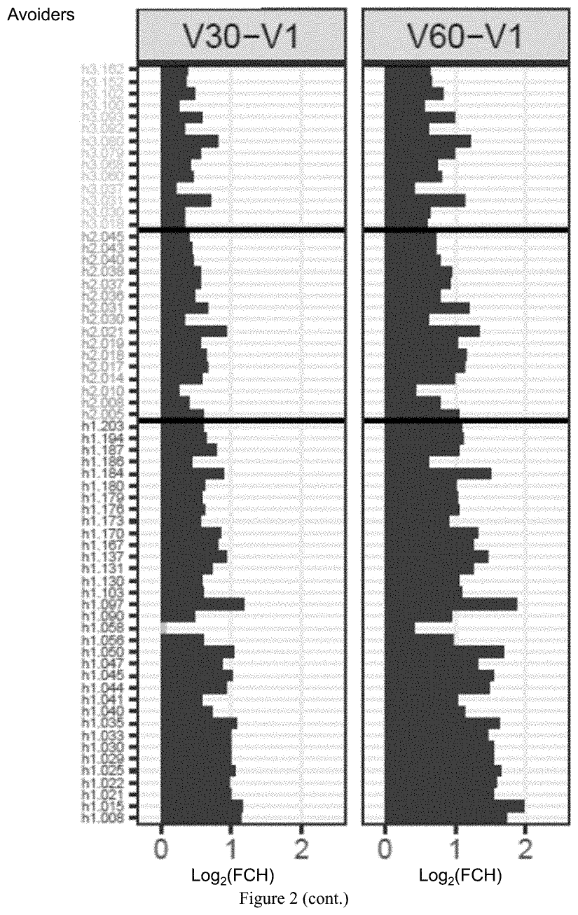

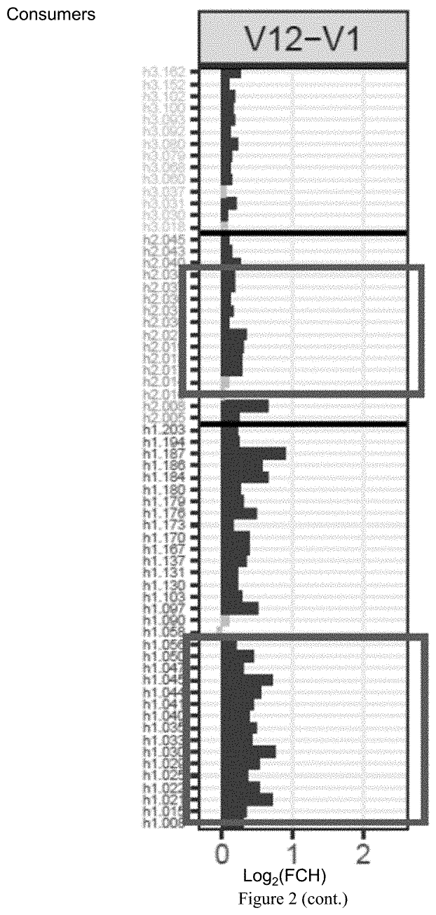

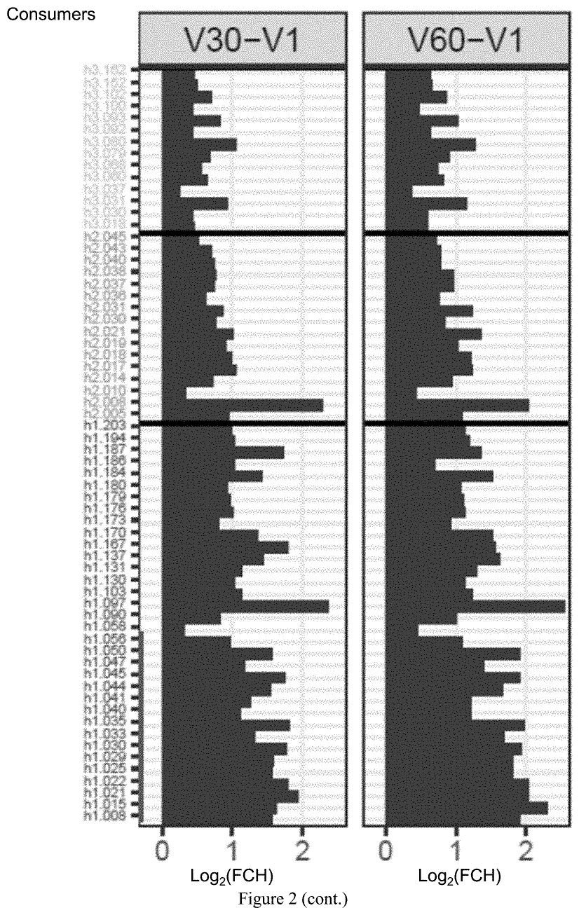

To assess IgG4 epitope differences between study groups (Avoiders and Consumers), the fold change in each epitope value across study visits compared to the baseline visit at the beginning of the study (V12-V1, V30-V1, and V60-V1) was determined. The results are plotted by epitope and summarized in FIG. 2. AT V30, IgG4 ws present at all epitopes in both groups, but greater in Consumers. Consumers generate IgG4 instead of IgE to relevant allergic epitopes very early, while Avoiders eventually produce IgG4, but after IgE. Avoiders also produce IgG4 due to non-oral exposure, such as association with environmental exposure (e.g., allergen in dust and patient questionnaires). In sum, the results indicate that IgG4 is present at all epitopes by V30 but demonstrates overall greater binding in the Consumer group. Further, the Consumer group developed IgG4 at the important IgE epitopes very early (V12) while the Avoiders produce IgG4 at these same epitopes but only after IgE has already been produced.

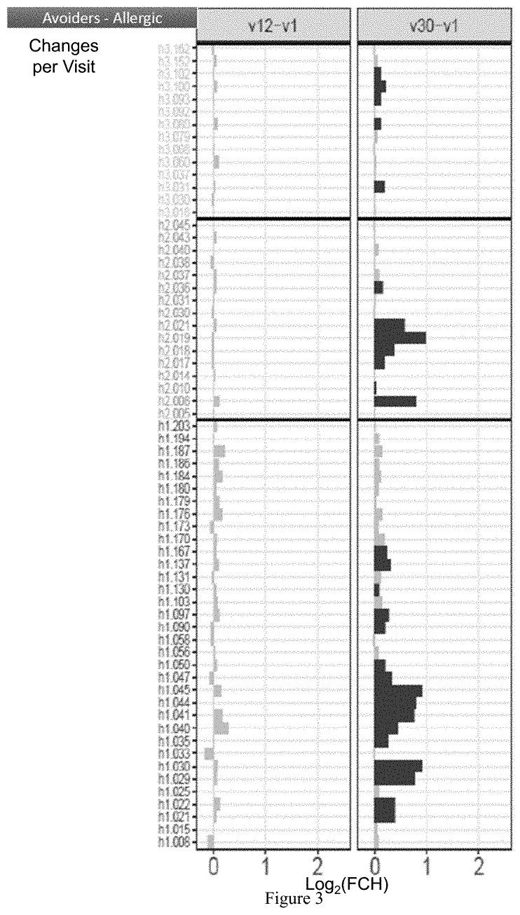

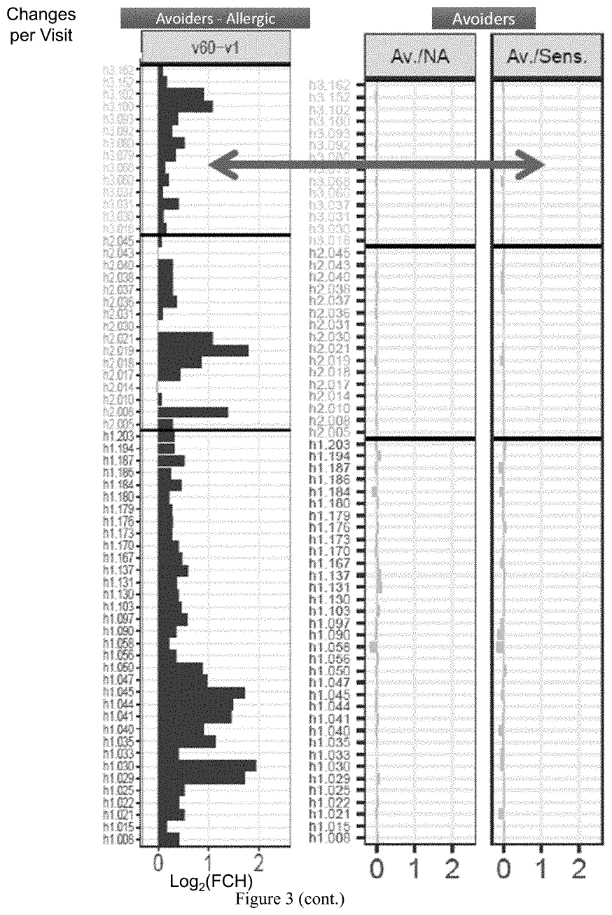

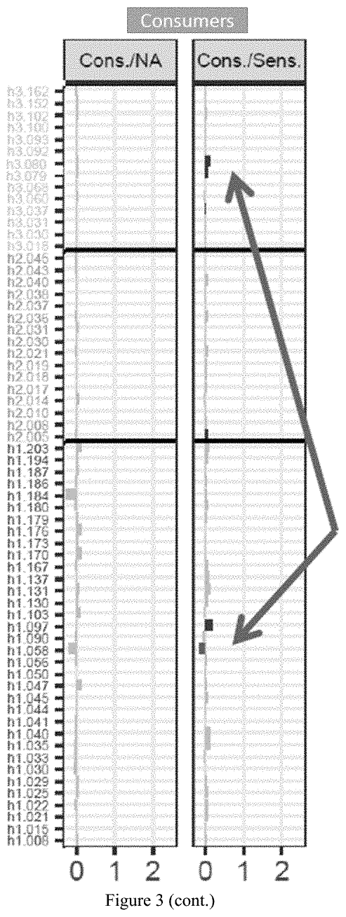

Since not all Avoiders became allergic, the epitope reactivity was compared between those allergic Avoiders and the non-allergic Avoider. The results are summarized in FIG. 3. No treatment differences were observed between Non-Allergic and Sensitized groups at any Visit. Epitopes bound by the Sensitized subjects were primarily different than those that became allergic. Few peptides had significant differences in the treatment changes over time only for Sensitized subjects. In sum, the results indicate that all of the IgE epitope specific reactivity was due to the Allergic Avoider and not the non-allergic avoiders. Further, as summarized in FIG. 3, sensitized patients have only a few IgE epitopes that change by V60 and these changes are different than those observed in the allergic group.

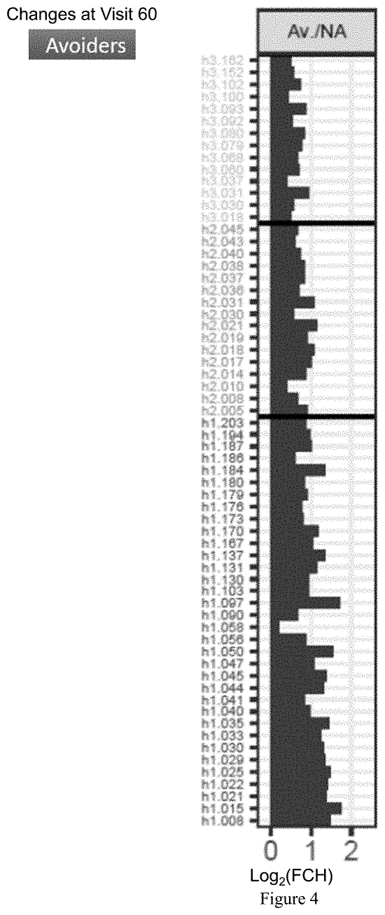

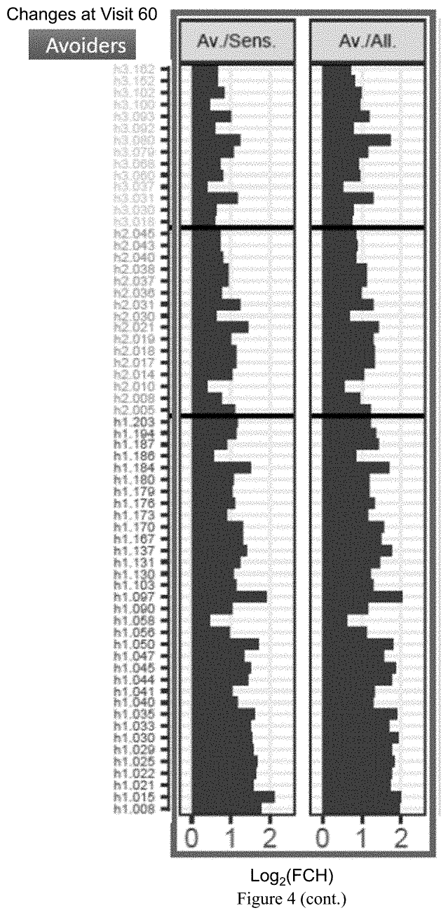

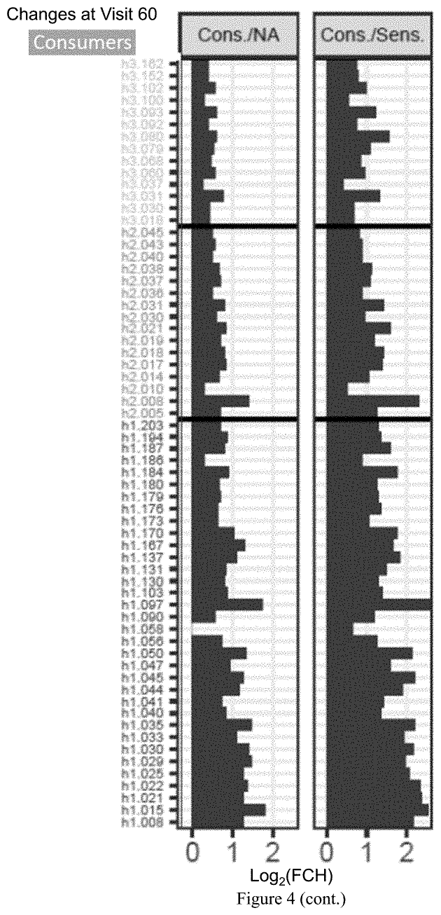

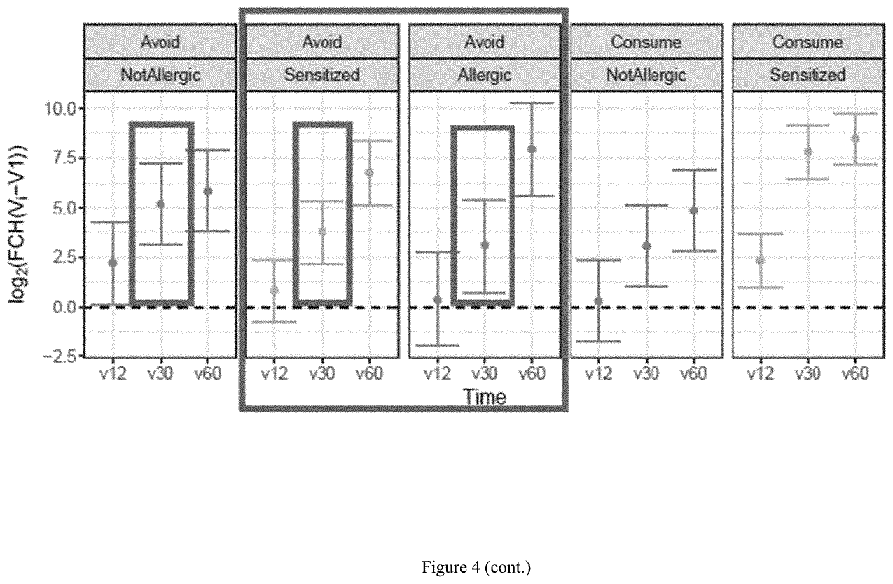

An additional examination of the IgG4 epitope binding is summarized in FIG. 4. Non-allergic subjects who avoid peanut consumption made more IgG4 earlier (V12 and V30) than either Allergic or Sensitized. There are no significant differences between Sensitized and Allergic, suggesting IgG4 is not protective. Among patients who are sensitized at V60, peanut consumption led to an earlier development of epitope-specific IgG4, especially in the two regions where allergic subjects develop IgE. In sum, while profound changes occur in all groups at year 60, non-Allergic Avoiders made more IgG4 earlier (V12, V30) than allergic or sensitized patients. However, there is not overall difference between the IgG4 response in sensitized verses allergic patients suggesting that IgG4 is not likely protective.

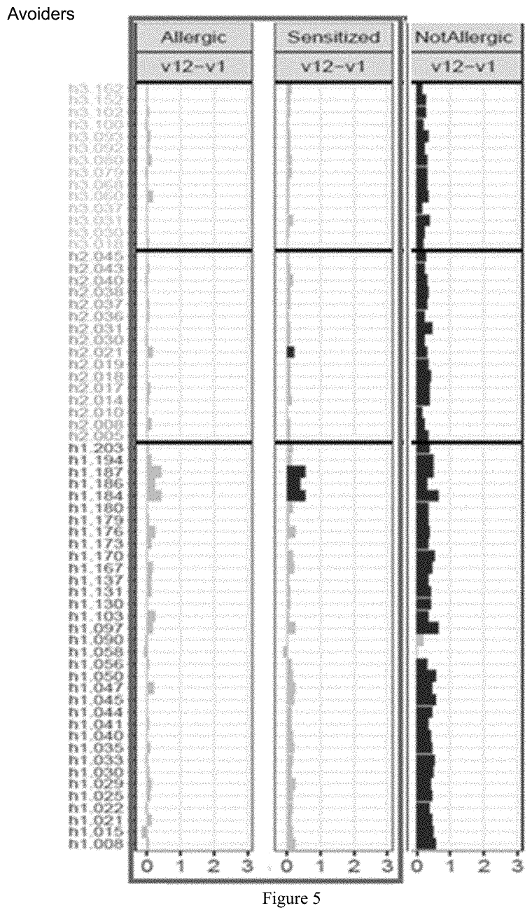

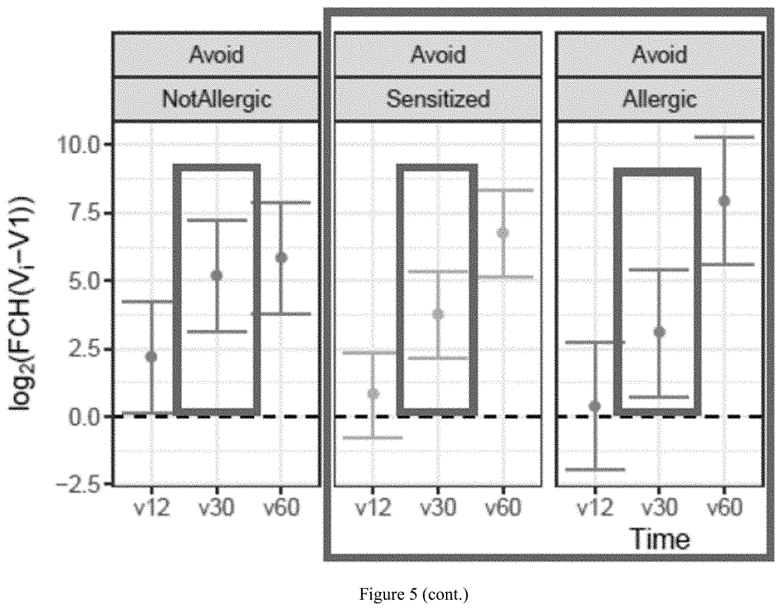

While the IgG4 response at V60 does not appear protective after 5 years of age, non-allergic patients in the Avoider group made more IgG4 early (V12 and V30) compared to allergic or sensitized patients. In addition, there was no significant differences between the IgG4 response for the Sensitized and Allergic patients, suggesting that IgG4 is not protective in the overall allergy response. The results are summarized in FIG. 5.

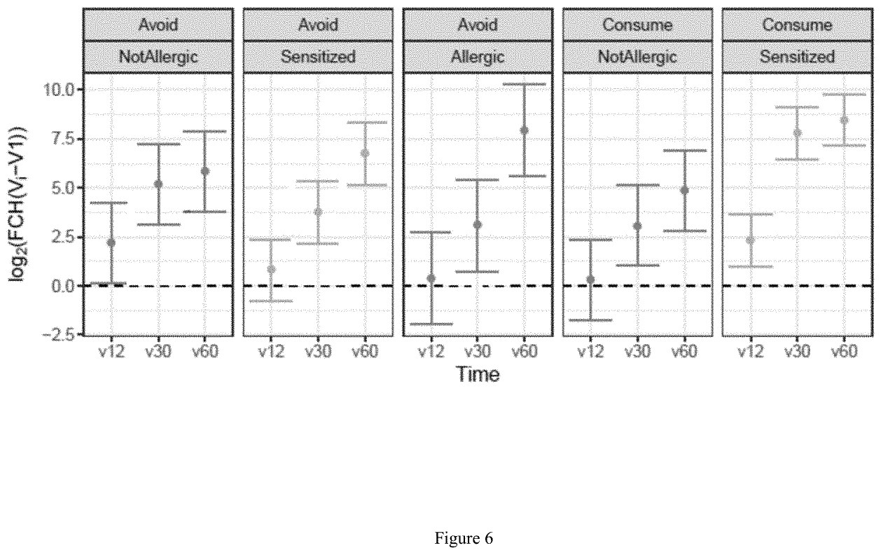

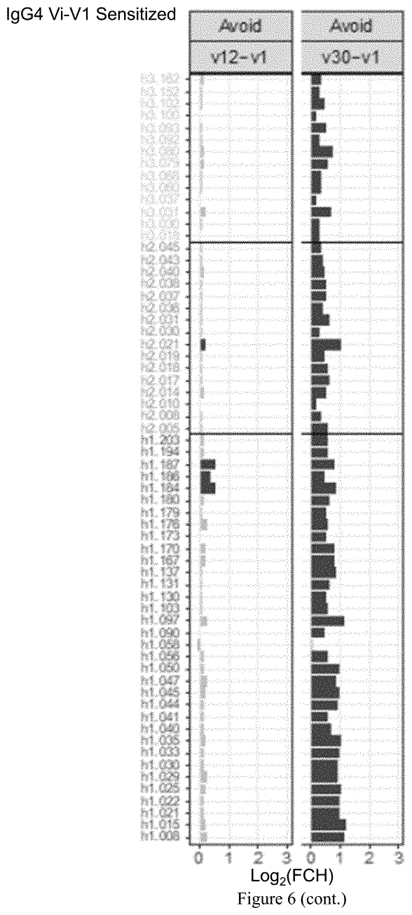

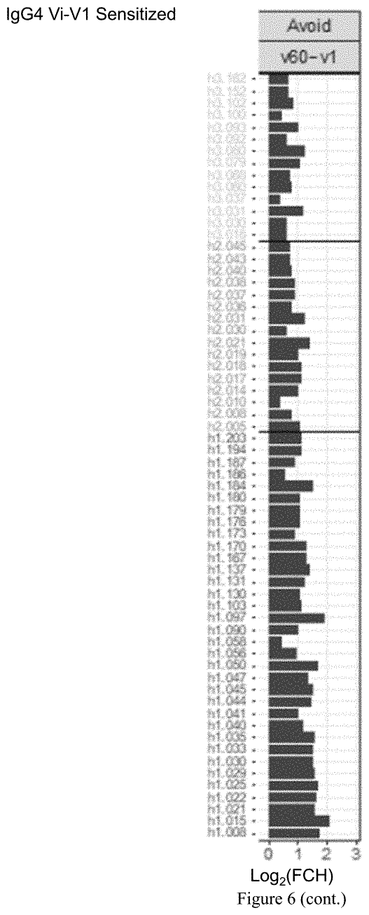

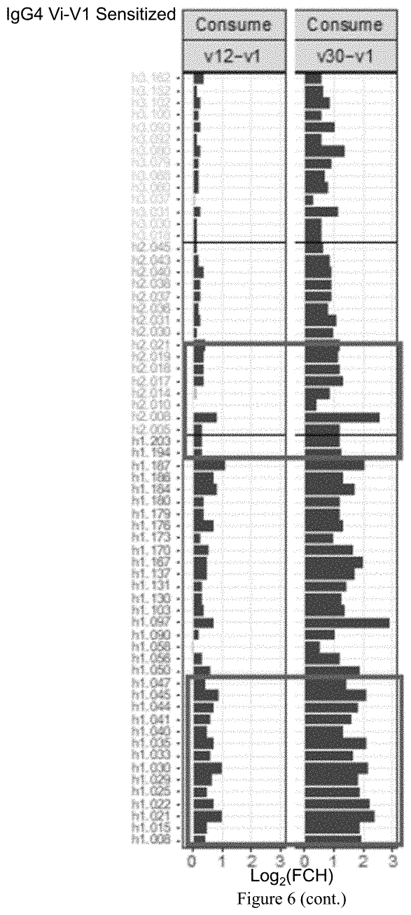

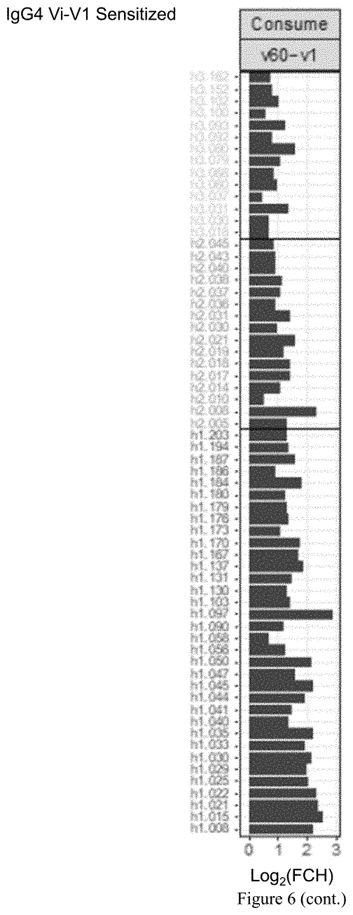

Consumption, however, did lead to an early (V12 and V30) IgG4 epitope expansion in Consumers who were sensitized, as summarized in FIG. 6, compared to Avoiders. This early expansion of IgG4 was especially observed at the regions of maximally observed IgE reactivity in Allergic patients. In sum, among patients who are sensitized at V60, peanut consumption led to an earlier development of epitope-specific IgG4, especially in the two regions where allergic patients develop IgE antibodies. These results suggest a possible early role for IgG4 in offsetting an allergy response.

These data, when taken together, suggest that an IgE response and possibly a IgG4 response at specific peptide may be a useful tool in diagnostics or therapeutic response monitoring in patients being treated by one or more immunotherapy approaches such as oral, sublingual, intradermal, sub-cutaneous, inhaled, epicutaneous, or a combination of methods to assess proper dose, therapeutic progress, adverse reactions, and successful outcome. The data also suggest that it may be possible to follow a patient after therapy to determine if food tolerance is maintained or if therapy would need to be started again.

The analysis used herein is a linear regression analysis commonly employed by those skilled in the art of allergies. Briefly, linear regression is a standard statistical approach to building a linear model that fits observations (e.g., allergic and non-allergic cases) to variables (e.g., epitope and IgE measurements). For example, the application of the linear regression methodology using Matlab version R2015b results in the following model build, where x1, x2, and x3 represent IgE and IgG4 epitope variables.

Generalized Linear regression model:

logit(y)˜1+x1+x2+x3
Distribution=Binomial

Estimated Coefficients:

| | Estimate | SE | tStat | pValue |
|---|---|---|---|---|
| (Intercept) | −1.6413 | 0.46258 | .5482 | 0.00038789 |
| x1 | 2.8755 | 0.84212 | 3.4146 | 0.00063878 |
| x2 | −0.33228 | 0.23678 | −1.4033 | 0.16053 |
| x3 | 0.21082 | 0.35729 | 0.59005 | 0.55516 |

133 observations, 129 error degrees of freedom

Dispersion: 1

Chi²-statistic vs. constant model: 25.2, p-value=1.42e-05 Thresholds are chosen to optimize the negative predictive value of the resulting linear regression model. Units for the IgE threshold are (KU/L). The threshold for the logistic regression model for more than one epitope is unitless.

### Example 2: Assessment of Epitope Classifiers for Prediction of Peanut Allergy

To assess the diagnostic applicability of the IgE and IgG4 epitope reactivity using this same (LEAP) cohort, patients whose final diagnosis was confirmed by OFC were selected to develop algorithms and identify classifiers for the prediction of allergy.

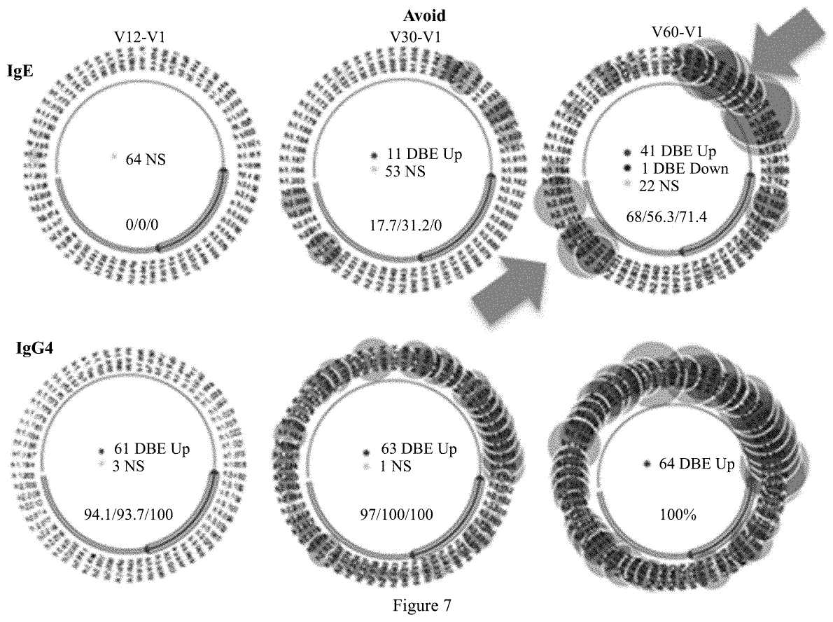

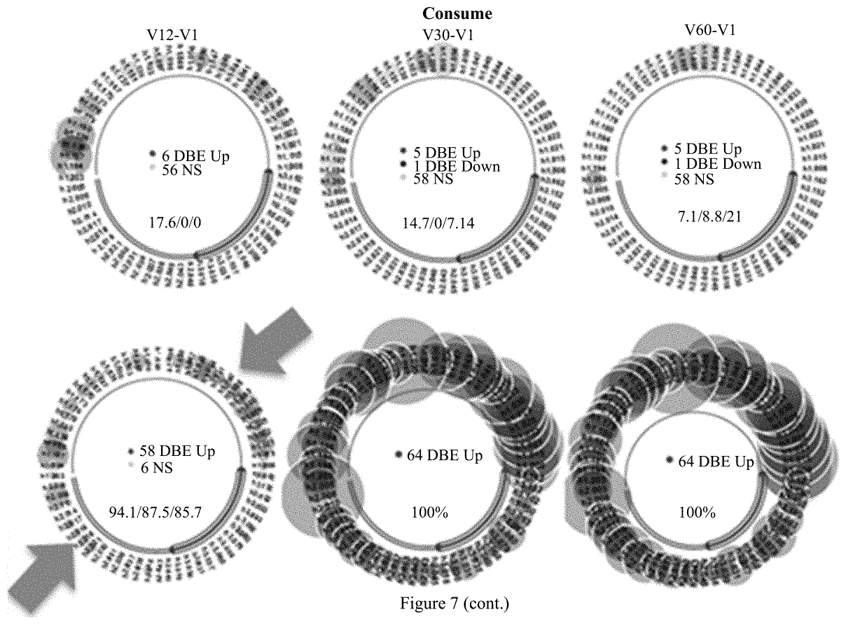

Using samples from 341 children at high risk of peanut allergy enrolled in the LEAP trial, the IgE/IgG4 epitope-specific binding over 5 years was evaluated (FIG. 7). IgE epitope-specific antibodies developed in patients in the Avoidance group, and were specific for those who have peanut allergy at 5 years of age and are predominantly in two regions (see FIG. 7, green arrows, left panel). IgG4 epitope-specific antibodies increased in all patients, suggesting that peanut exposure is occurring via non-oral routes in peanut Avoiders. In particular, peanut Consumers developed IgG4 early (see, FIG. 7, right panel), especially in the relevant regions (see, green arrow), diverting to a “protective” IgG4 response instead of IgE, while peanut Avoiders eventually developed IgG4 antibodies in addition to IgE. The assay has been validated for peanut and determined similar excellent ICC values >0.90 for all peanut-specific epitopes (>0.95 in the majority) in a previous COFAR cohort of PA patients and in the LEAP cohort. The results indicate that early consumption or avoidance of peanuts induces changes in the IgE epitope repertoire that are associated with intervention outcome.

Epitopes with Top AUC Performance:

Cases and controls were defined as OFC-confirmed allergic and non-allergic patients, respectively. All IgE epitopes were then assessed individually as a classifier for prediction of being allergic or non-allergic. AUC was used as the performance metric. The top 10 performing epitopes (allergic epitopes) appear in Table 2.

TABLE 2

| IgE Analyte | AUC | Epitope |
|---|---|---|
| 58 | 0.73 | Ara h 2.008 |
| 27 | 0.68 | Ara h 1.047 |
| 41 | 0.67 | Ara h 1.167 |
| 10 | 0.65 | Ara h 1.022 |
| 21 | 0.65 | Ara h 1.040 |
| 45 | 0.64 | Ara h 1.176 |
| 92 | 0.64 | Ara h 3.102 |
| 25 | 0.64 | Ara h 1.045 |
| 52 | 0.64 | Ara h 1.194 |
| 38 | 0.64 | Ara h 1.131 |

Integrated Tests with TOP AUC Performance:

Cases and controls were defined as OFC-confirmed allergic and non-allergic patients, respectively. Panels of IgE and/or IgG4 epitopes were assessed as classifiers for prediction of being allergic or non-allergic. AUC was used as the performance metric. In addition, examples of panels that also incorporated other clinical measurements such as SPT results, specific peanut IgE (sIgE) measures, and peanut whole protein component (Ara h2) were also included. Panels were formed by using standard linear regression methodology. The notation [A B C] is used to indicate that the panel of epitopes and/or clinical measurements A, B, and C were integrated into a panel by linear regression. The use of linear regression to assess a combination of factors (peanut peptides and/or other factors described herein) is well known the those skilled in the art. Representative examples of such combinations include, but are not limited to:

[IgE_Ara h 2.008, IgG4_Ara h 2.021, IgG4_ara h 3.152] has AUC=77%

[IgE_Ara h 2.008, IgG4_Ara h 2.021, SPT] has AUC=81%

[IgE_Ara h 2.008, IgG4_Ara h 2.021, IgG4_ara h 3.152, sIgE] has AUC=81%

[IgE_Ara h 2.008, IgG4_Ara h 2.021, IgG4_ara h 3.152, Ara h2] has AUC=75%

In general, an AUC for any combination of peanut peptides in combination with any other factors that is less than 50% is indicative of a non-allergy status, whereas an AUC that is greater than or equal to 50% is indicative of an allergy status. In some embodiments, an AUC for any combination of peanut peptides in combination with any other factors that is less than 55% is indicative of a non-allergy status, whereas an AUC that is greater than or equal to 55% is indicative of an allergy status. In some embodiments, an AUC for any combination of peanut peptides in combination with any other factors that is less than 60% is indicative of a non-allergy status, whereas an AUC that is greater than or equal to 60% is indicative of an allergy status. In some embodiments, an AUC for any combination of peanut peptides in combination with any other factors that is less than 65% is indicative of a non-allergy status, whereas an AUC that is greater than or equal to 65% is indicative of an allergy status. In some embodiments, an AUC for any combination of peanut peptides in combination with any other factors that is less than 70% is indicative of a non-allergy status, whereas an AUC that is greater than or equal to 70% is indicative of an allergy status.

### Hierarchical Integrated Test (Prophetic)

The methodology employed in this example is the same as in the above sections except that the classification is performed in a hierarchical fashion. First, the sIgE measurement is used to identify non-allergic cases. For those patients not classified by sIgE, the logistic regression panel [IgE_Ara h 2.008, IgG4_Ara h 2.021, IgG4_ara h 3.152] is then used to classify the remaining patients as either allergic or non-allergic. If sIgE≤0.03 kU_{A}/L, then the subject is “Not Allergic.” If [IgE_Ara h 2.008, IgG4_Ara h 2.021, IgG4_ara h 3.152]≤0.20, then the subject is “Not Allergic. Otherwise, the subject is “Allergic.” Thresholds in this hierarchical classifier are used to make classification decisions. This combination of factors is used herein, for example, to eliminate false positives. These can be varied to achieve different classification results. In the example provided, the sensitivity and specificity of the hierarchical classifier are 90% and 54%, respectively.

### Example 3: Trial

Based on the strong data presented herein, it is expected that the pattern of IgE/IgG4 binding to peanut epitopes will be highly informative and useful in characterizing the severity of a patient's allergy disease, assessing patients longitudinally to guide dosing initially and during the study, tracking or predicting adverse events during the study (to improve safety), confirming a patient's allergy status at the clinical end-point of therapy, and monitoring a patient's post therapy to determine if/when additional therapy might be necessary to maintain therapeutic responsiveness.

A subset of patient samples will be identified. Minimally, the samples will include both Ar101-treated and placebo arms including patients that: 1) remained in the study through the clinical end-point; 2) continued to be monitored after the clinical end-point to assess sustained unresponsiveness; 3) dropped out of the study due to an adverse reaction; 4) dropped out of the study without an adverse reaction; 5) have serum component and OFC data available at multiple time points longitudinally during the study; and 6) fall within different dosing schedule groups or were escalated at different rates during the study.

Plasma or serum samples from patients enrolled in the trial taken at various time points after initiation of OIT will be assayed blinded to clinical information related to the trial. Samples will be assayed for IgE, IgG4, and IgA epitope binding using the methods described herein (e.g., 64-plex peanut (ara h1, h2, and h3) epitope test).

Epitope Profiling (Bead-Based Epitope Assay (BBEA):

For epitope mapping, the present methods subdivide the proteins found in specific foods into smaller peptides, or compounds consisting of two or more amino acids. For epitope differentiation, the mapped peptides are separately attached onto beads to allow for high-throughput analysis and epitope binding assessment. For epitope pattern determination, the reactivity of the patient's IgE response is determined. In the reaction, antibodies attach to peptide beads and the test is designed to isolate and determine individualized reactions per peptide so clinicians can get a more accurate and complete picture of a patient's allergy profile. The results are “mapped” with individual peptide results, creating a therapy response profile used to classify patients.

In brief, for the epitope assay, peanut peptides (CS Bio, Menlo Park, Calif., USA) were coupled to LumAvidin beads (Luminex Corporation, Austin, Tex.) and stored in PBS-TBN buffer (lx PBS+0.02% Tween20+0.1% BSA). A master mix of peptide-coupled beads was prepared in PBS-TBN buffer and 100 μL of the bead master mix was added to filter plates. After washing the beads, 100 μL of subject's plasma at 1:10 dilution was added to the triplicate wells. The plates were incubated on a shaker for 2 hours at 300 rpm at room temperature. Excess plasma was removed and the plate was washed. 50 μL/well of mouse anti-human IgE-PE (Thermo-Pierce Antibodies, Clone BES, diluted 1:50 in PBS-TBN) or mouse anti-human IgG4 Fc-PE (SouthernBiotech, Clone HP6025, diluted 1:400 in PBS-TBN) secondary antibody was added and the plates were incubated for 30 minutes. After a final wash, 100 μL of PBS-TBN buffer was added to each well to re-suspend the beads, which were then transferred to fixed-bottom 96-well reading plates, and quantified on the Luminex 200 instrument (Luminex® 100/200™ System, Luminex Corporation, Austin, Tex.).

All samples were processed in triplicates. To eliminate background intensity, a buffer sample (PBS-TBN buffer) was also processed in triplicates in each plate. The median fluorescence intensity (MFI) for each epitope and sample was obtained directly from the Luminex reader's output. For each sample i, and epitope j, the binding measurements Bᵢⱼ was defined as:

$${Y}_{ij} = {log}_{2} ( {MFI}_{ij} + 0.05 ) ; {B}_{ij} = {Y}_{ij} - \frac{1}{{N}^{n s}} {\sum }_{k = 1 ; n s} {Y}_{kj}$$

where ns represents the non-specific binding (buffer) samples.

### Example 4: Study Cohort

Plasma samples of peanut non-allergic and allergic patients from CoFAR2 prospective pediatric cohort were used in the analysis. Allergy diagnosis at each visit was defined as: 1) Allergic (serologic: peanut sIgE>14 kU_{A}/L; confirmed: OFC+ or (convincing history+serologic)); 2) Non-Allergic (sensitized: tolerant but peanut sIgE>0.35 kU_{A}/L; not sensitized: tolerant and no evidence of peanut sIgE). Three visits were scheduled: 1) Baseline (about 0 years); 2) Visit 2 (about 2 years); and 3) Visit 5 (about 4+ years). Baseline information is present in Table 3.

TABLE 3

| | | |
|---|---|---|
| Non-Allergic | Serologic | Confirmed |

Baseline (N = 141)

| | |
|---|---|
| 40% (n = 57) | 60% (n = 84) |

V2 (N = 129)

| | | |
|---|---|---|
| 54% (n = 70) | 39% (n = 60) | 7% |

Baseline (N = 141)

| | | |
|---|---|---|
| 48% (n = 89) | 32% (n = 59) | 20% |

Previous studies showed that age (↑ younger), gender (↑ males), race (↑ African American), and the extent and history of allergic reactions are predictive of peanut allergy. In the present cohort, Allergic and Non-Allergic groups were comparable among those predictors at baseline. Since CoFAR enrolls pediatric patients at high risk for peanut allergy, the prevalence of AD is very high in both groups (90% and 98%). The baseline information of patients is shown in Table 4.

TABLE 4

| | Non-Allergic | Allergic | |
|---|---|---|---|
| | n = 57 | n = 84 | P-value |
| Age | 0.84 (0.28) | 0.83 (0.25) | 0.71 |
| Sex = Male (%) | 41 (71.9) | 65 (77.4) | 0.591 |
| Race (%) | | | 0.14 |
| Asian | 1 (1.8) | 10 (11.9) | |
| African American | 10 (17.5) | 14 (16.7) | |
| White | 45 (78.9) | 57 (67.9) | |
| Other | 1 (1.8) | 3 (3.6) | |
| Non-Hispanic/Latino | 54 (94.7) | 80 (95.2) | 1 |
| origin (%) | | | |
| Weight | 9.14 (1.54) | 8.57 (1.29) | 0.02 |
| Height | 72.09 (4.83) | 70.92 (4.67) | 0.153 |
| SPT Score (mm, | 0.58 (1.43) | 9.04 (5.20) | <0.001 |
| Peanut) | | | |
| Specific IgE | 1.63 (8.89) | 35.48 (45.09) | <0.001 |
| (KU_{A}/L) | | | |
| Specific IgG | 5.44 (10.57) | 20.26 (26.52) | <0.001 |
| (mgA/L) | | | |
| Specific IgG4 | 0.09 (0.47) | 0.36 (0.67) | 0.011 |
| (mgA/L) | | | |
| Ratio sIgG4/sIgE | 4.43 (3.90) | 1.48 (2.62) | <0.001 |
| (KU_{A}/L, log2) | | | |
| IgE to Peanut | | | |
| Components | | | |
| Ara h 1 (KU_{A}/L) | 0.43 (2.59) | 7.59 (18.71) | 0.006 |
| Ara h 2 (KU_{A}/L) | 1.94 (12.53) | 21.75 (45.12) | 0.002 |
| Ara h 3 (KU_{A}/L) | 0.14 (0.69) | 3.57 (8.28) | 0.003 |

At baseline, all allergic patients are “Serologic”

Over time, more allergic children showed IgE binding to a greater number of epitopes. Clear differences in IgE epitope repertoire were observed between Allergic and Non-Allergic children across all visits. IgG4 increased with age across all outcomes with very similar IgG4 profiles between the two groups at visit 5. FIG. 8 shows results.

Several models have been developed regarding epitopes including, for example, Random Forest (RF) and Cross-Validation (CV). RF models performed well in cross-validation, especially for children at ages 2 and 4+(see, Table 5). The RF model can correctly predict allergy diagnosis in the training set in almost 100% of patients (see, Table 6). CV is a measure of how well the model did in all training iterations, and is considered as an unbiased assessment of a predictive model (see, FIG. 9).

TABLE 5
Cross-Validation (RF)

| Metric | V0 | V2 | V5 | All |
|---|---|---|---|---|
| AUC | 0.90 | 0.99 | 0.97 | 0.95 |
| Sensitivity | 82% | 95% | 89% | 89% |
| Specificity | 83% | 96% | 92% | 90% |

TABLE 6
Training (RF)

| | V0 | V2 | V5 | V0 |
|---|---|---|---|---|
| | n = 106 | n = 98 | n = 139 | n = 343 |
| Metric | alg = 63 | alg = 45 | alg = 72 | alg = 180 |
| AUC | 0.98 | 1 | 1 | 1 |
| Accuracy | 93% | 100% | 100% | 100% |
| Sensitivity | 82% | 100% | 100% | 100% |
| Specificity | 100% | 100% | 100% | 100% |
| PPV | 100% | 100% | 100% | 100% |
| NPV | 85% | 100% | 100% | 100% |

alg = allergic

The Epitope Model was carried out in actual testing. Testing data had: 1) 35 (21 allergic) patients at V0; 2) 31 (14 allergic) patients at V2; and 3) 46 (24 allergic) patients at V5. The AUC in testing provided the following results: 1) 0.70-11 misclassified—at V0; 2) 0.88-4 misclassified—at V2; 3) 0.84-7 misclassified—at V5; and 4) 0.84-18 misclassified—at all visits. Performance metrics in testing are depicted in FIG. 10.

The performance of the epitope models in testing data was compared with allergy diagnosis based on the ImmunoCAP data. ImmunoCAP diagnosis was “Allergic” if a patient had positive (≥0.35 kU_{A}/L) sIgE to peanut, Ara h1, Ara h2, and Ara h3. For every visit (and all visits combined), the epitope-based models outperformed the component-based allergy diagnosis (see, FIG. 11). Only 10 patients in the test data had OFC-confirmed diagnosis, both epitope and ImmunoCAP correctly diagnosed 8/10 children.

The relationship between epitopes and sIgE to peanut was investigated. Children younger than 2 years of age are more likely to develop antibodies to the whole peanut extract than to the component proteins or their linear epitopes. In addition, including sIgE to the whole peanut might be instrumental in identifying allergic children at a very young age. AUC in CV is depicted in FIG. 12. As expected, for the 0 years model, sIgE was the top predictor and model performance in CV improved, with AUC reaching 0.98 (compared to 0.89). The AUC for visits 2 and 5 remained the same.

Three sets of predictors were used to predict allergy outcome: 1) epitopes; 2) epitopes+peanut-specific IgE; and 3) Peanut-specific+component-specific IgE: sIgE to peanut, Ara h1, Ara h2, and -Ara h3. IgE antibodies to epitopes alone were able to accurately diagnose peanut allergy in the majority of patients, and did better than components (see, Table 7 and FIG. 13). Adding sIgE to peanut further improved performance of the epitope models in CV and in testing, with 95% (107/112) of patients correctly diagnosed.

TABLE 7
Epitopes + sIgE in Testing

| Metric | V0 | V2 | V5 | All |
|---|---|---|---|---|
| AUC | 1 | 0.94 | 0.95 | 0.95 |
| Sensitivity | 100% | 100% | 96% | 94% |
| Specificity | 100% | 88% | 95% | 95% |

Epitope profiles show promising performance as predictive biomarkers for diagnosing peanut allergy. Using epitopes alone, 83% of children at any visit were accurately identified as “Allergic” compared to 70% when using ImmunoCAP standard guidelines. Both epitopes and component testing performed better when identifying peanut allergy in children at 2 years visit or later. Adding sIgE to whole peanut to epitope models markedly improved the diagnostic model performance with an accuracy >95% in testing data.

### Example 5: Discovery and Validation of Peanut Allergy Diagnostic

### Methods

Discovery: Discovery of the test was performed on 133 subjects (31 allergic, 102 non-allergic) from the avoidance arm of the LEAP study. All diagnoses were determined by OFC at age 5 years. Plasma samples were obtained at years 2.5 and 5 for each subject. These samples were analyzed using the BBEA methodology described above to obtain the IgE and IgG4 epitope levels for each subject at year 2.5 and year 5. The IgE (IgG4) epitope levels for each subject were normalized by the median value of all IgE (IgG4) epitope measurements.

Data was analyzed at year 5 to determine the best performing IgE or IgG4 epitope for segregating allergic and non-allergic subjects. Specifically, the best performing IgE or IgG4 epitope is the one with the best AUC for classifying those subjects as allergic or non-allergic after initial triage of subjects by peanut specific IgE (sIgE) level below 0.1 kU/L. It was then confirmed that this same IgE or IgG4 epitope was also the best performing epitope at year 2.5.

After identification of the best performing epitope and decision thresholds, the diagnostic test was fully locked down prior to validation.

Validation:

Validation of the test was performed on 81 subjects (23 allergic, 58 non-allergic) from the CoFAR2 study. All diagnoses were determined by OFC at age 5 years. Plasma samples were obtained at years 2 and 5 for each subject. These samples were analyzed using the BBEA methodology described above to obtain the IgE and IgG4 epitope levels for each subject at year 2 and year 5. The epitope levels for each subject were normalized by the median value of all IgE (IgG4) epitope measurements. All data remained blinded until the diagnostic test was fully locked down.

Validation of the diagnostic test was performed using predefined hypotheses and thresholds. First, the performance of the diagnostic test using threshold 0.1 Ku/L for sIgE and threshold 0.30 for the optimal IgE (IgG4) epitope was statistically significant using the chi-squared test for association for subjects at year 5. Similarly, the performance of the diagnostic test for subjects at year 2 was assessed. All data analyses performed using Matlab R2015b.

The integrated test algorithm, where various values for threshold T were validated, that was used included an initial query: Is the sIgE≤0.10? If the answer to this initial query is “yes”, then the conclusion of “not allergenic” is achieved. If the answer to the initial query is “no”, then a follow-up query is requested: Is the IgE h2.008≤T? If the answer to this follow-up query is “yes”, then the conclusion of “not allergenic” is achieved. If the answer to the follow-up query is “no”, then the conclusion of “allergenic” is achieved. Units for sIgE are kU/L and the IgE h2.008 measurement is unitless.

### Results

Discovery: Table 8 presents the performance of the top three IgE and/or IgG4 epitopes in the LEAP cohort, both on their own and also after triage by sIgE at threshold 0.1 kU/L. Data is presented at years 2.5 and 5.

TABLE 8
Top performing epitopes both individually and in combination
with sIgE where AUC is used as the metric

| | Year 2.5 | | | | Year 5 | | | |
|---|---|---|---|---|---|---|---|---|
| | Epitope | | sIgE and | | Epitope | | sIgE and | |
| | Alone | | Epitope | | Alone | | Epitope | |
| Rank | Epitope | AUC | Epitope | AUC | Epitope | AUC | Epitope | AUC |
| 1 | IgE | 72% | IgE | 77% | IgE | 71% | IgE | 69% |
| | h2.008 | | h2.008 | | h2.008 | | h2.008 | |
| 2 | IgE | 68% | IgE | 70% | IgE | 69% | IgE | 69% |
| | h1.179 | | h1.022 | | h1.173 | | h1.179 | |
| 3 | IgE | 68% | IgG4 | 67% | IgE | 69% | IgG4 | 69% |
| | h1.022 | | h1.025 | | h2.021 | | h3.080 | |

The discovery data indicate that epitope IgE h2.008 has optimal performance both at years 2.5 and 5. Furthermore, the same epitope has optimal performance both alone and in combination with sIgE.

Validation:

The demographics of the LEAP and CoFAR2 studies are described in, for example, Toit et al., N. Engl. J. Med., 2015, 372, 803-813 and the world wide web at “leapstudy.co.uk/” (LEAP) and Sicherer et al., JACI, 2016, 137, AB152 and the world wide web at “clinicaltrials.gov/ct2/show/NCT00356174” (CoFAR2).

FIG. 14 presents a ROC plot comparing the performance of components h1, h2, h3, epitope IgE h2.008, sIgE, and combinations with sIgE.

Table 9 presents the performance of individual measures and those integrated with sIgE. The integrated test (sIgE+h2.008) is presented for various illustrative values of threshold T.

TABLE 9

| Diagnostic | AUC | Sensitivity | Specificity | NPV | PPV |
|---|---|---|---|---|---|
| h1 | 64% | 78% | 40% | 82% | 34% |
| h2 | 74% | 83% | 43% | 86% | 37% |
| h3 | 52% | 91% | 9% | 71% | 28% |
| IgE h2.008 | 84% | 91% | 53% | 94% | 44% |
| sIgE | 77% | 91% | 36% | 91% | 36% |
| sIgE + h2 | 80% | 91% | 40% | 92% | 38% |
| sIgE + h2.008 (T = .30) | 88% | 91% | 79% | 96% | 64% |
| sIgE + h2.008 (T = 1.2) | 88% | 70% | 97% | 89% | 89% |
| sIgE + h2.008 (T = 2.0) | 88% | 48% | 98% | 83% | 92% |

In this example, the integrated test is a strong rule out test (T=0.30) with a NPV of 96%. Conversely, the integrated test is a strong rule in test (T=2.0) with a PPV of 92%. At T=1.2 the integrated test is both a strong Rule In and Rule Out test.

At year 2, the performance of sIgE+Ige_h2.008 is stable with an AUC, sensitivity, specificity, NPV, and PPV of 88%, 96%, 71%, 98% and 56%, respectively. The statistical significance of IgE h2.008 on its own or in combination with sIgE has p-value <0.0001 at years 2 and 5.

### Example 6: Multiple Epitope Classifiers

### Methods

IgE epitopes were assessed in the LEAP and CoFAR2 sample sets in terms of the following criteria: 1) reproducibly high performance (AUC) across both studies; and 2) frequency of participation on high performing epitope panels (using logistic regression models) of size 2, across both studies.

### Results

Using the criteria listed above, the epitopes list in Table 10 below are the most reproducible epitopes that perform well on multiple epitope panels. Presented are their performances as univariate predictors but also in combination with IgE h2.008 using a logistic regression fit.

TABLE 10

| | | Performance (AUC) |
|---|---|---|
| | Performance (AUC) | In Combination with |
| Epitopes (IgE) | in CoFAR2 | IgE h2.008 |
| H2.008 | 78% | 78% |
| H2.021 | 64% | 89% |
| H2.017 | 61% | 87% |
| H2.019 | 60% | 88% |
| H3.093 | 61% | 80% |
| H1.173 | 67% | 79% |
| H2.040 | 75% | 89% |

In forming high performance multiple epitope diagnostic classifiers, the epitopes in Table 11 are the optimal ones from which to select.

### Example 7: Multiple Biomarker Classifiers

### Methods

IgE epitopes in the LEAP and CoFAR2 sample sets were combined with Skin Prick Testing (SPT) and peanut specific IgE (sIgE) were assessed in terms of the following criteria: 1) reproducibly high performance (AUC) across both studies, and 2) frequency of participation on high performing epitope panels (using logistic regression models) and SPT was reported relative to wheal size.

Intended Use Case:

The intended use case is a blood test that would be ordered after a SPT has been performed. The result of the SPT (wheal size) would be provided on the test request form.

Intended Use:

The intended use of the blood test is to determine, with high probability, if the subject tested has a peanut allergy or not.

Single Threshold Test:

The single threshold test is as follows: If sIgE≤0.10, or SPT≤T1, or h2.008≤0.8, or h2.010≤T2, then the subject is not allergic. Otherwise, the subject is allergic.

Essentially, the test states that a subject must have a conformational epitope hit (sIgE), a positive SPT, and multiple linear epitope hits to be peanut allergic.

The performance of this test, depending on the choice of T1 and T2 is shown in Table 11.

TABLE 11

| T1 | T2 | Sens | Spec | NPV | PPV |
|---|---|---|---|---|---|
| 0 | 1.1 | 65% | 97% | 88% | 88% |
| 4 | 0.75 | 94% | 91% | 91% | 94% |

Double Threshold Test:

The double threshold test is as follows, where T2 and T3 are the lower and upper thresholds, respectively: If sIgE≤0.10, or SPT≤T1, or h2.008≤0.8, or h2.010≤T2, then the subject is not allergic. Otherwise, if h2.010≥T3, then the subject is allergic. Otherwise the test result is indeterminant.

The performance of this test, depending on the choice of T1, T2 and T3 is shown in Table 12.

TABLE 12

| T1 | T2 | T3 | % Indet* | NPV | PPV |
|---|---|---|---|---|---|
| 0 | 0.75 | 1.26 | 5% | 90% | 93% |
| 0 | 0.75 | 1.26 | 10% | 90% | 100% |
| 4 | 0.75 | 1.10 | 2% | 91% | 100% |

Note that when there are two thresholds, the sensitivity and specificity of a test cannot be specified. Double threshold tests are challenging for most individuals to interpret.
*The percentage of indeterminate cases is an estimate.

### Example 8: Random Forest Classifiers

### Methods

Utilizing a cohort of 318 subjects from the CoFAR natural history study, IgE and IgG4 binding to sequential allergenic peanut epitopes in high risk infants from 3-15 months to 8 years of age was measured and determined their utility to predict clinical peanut allergy. IgE and IgG4 antibody binding to sequential epitopes found on Ara h1-3 was assessed using a Luminex bead based assay. Sera from 318 subjects were evaluated for IgE and IgG4 epitope-specific antibodies at baseline, 2 and 5 years. Random Forest and machine learn algorithms were used to build models that could predict the outcome of their allergy status based on epitope-binding profiles (EBPs). Model performance was evaluated by resampling methods and verification using binded samples from the same cohort (30% of original set) and Accuracy, AUC, Sensitivity, and Specificity were obtained along with confidence intervals. Two hundred and twenty-five (225) subjects were randomly selected for “model development” and 93 for “testing”, for a total of 122 and 48 allergic patients at 5 years, respectively.

### Results

Using an Age-agnostic model and models specific for each age, the later was found to have achieved better results. IgE-profiles were sufficient to predict OFC outcome, while models with only IgG4 did not perform as well. The Age-agnostic model was not as accurate as the Age-specific models. Of strategies evaluated, the Random Forest algorithm with down bootstrap resampling performed best, with an average AUC>0.87 in cross-validation at 0 years, reaching 0.99 and 0.95 for OFCs conducted at 2 and 5 years. The final IgE-based model for each age group was then evaluated in the ‘unseen’ test data. Allergy status at baseline was accurately predicted in 76 patients (82% accuracy) with higher accuracy in allergy status at 2 and 5 years (91% and 87%, respectively). Although specificity was comparable across all age groups, models for the first year had a lower true positive rate. Since younger allergic patient tend to develop reactivity to non-liner, conformational epitopes, algorithms were also develop using peanut specific IgE as a classifier. Including peanut specific IgE (sIgE) dramatically improved the models compared to epitopes alone using a random forest algorithm and the performance as summarized in Table 13 below.

TABLE 13

| | Testing | | | | | | | |
|---|---|---|---|---|---|---|---|---|
| | Epitopes | | | | Epitopes + sIgE | | | |
| | (BF = 75, RF) (T1) | | | | (BF = 75, RF) (T2) | | | |
| | Visit | | | | | | | |
| | 0 | 2 | 5 | All | 0 | 2 | 5 | All |
| Accuracy | 0.68 | 0.87 | 0.84 | 0.83 | 1.00 | 0.94 | 0.95 | 0.95 |
| AUC | 0.70 | 0.88 | 0.84 | 0.83 | 1.00 | 0.94 | 0.95 | 0.95 |
| Sensitivity | 0.55 | 1.00 | 0.83 | 0.89 | 1.00 | 1.00 | 0.96 | 0.96 |
| Specificity | 0.86 | 0.76 | 0.86 | 0.77 | 1.00 | 0.88 | 0.95 | 0.94 |
| PPV | 0.85 | 0.78 | 0.86 | 0.81 | 1.00 | 0.88 | 0.96 | 0.95 |
| NPV | 0.57 | 1.00 | 0.82 | 0.87 | 1.00 | 1.00 | 0.95 | 0.96 |

In Table 13, the epitopes that appeared in at least 75% of top models “Bagging Frequency” as part of the Random Forest predictors T1 (without sIgE) and T2 (with sIgE) are listed below: T1 epitopes, in BF order: h2.008_IgEE, h2.008_IgEG4, h1.021_IgEE, h1.030_IgEE, h1.040_IgEE, h3.102_IgEE, h1.186_IgEG4, h1.015_IgEE, h2.010_IgEG4, h2.037_IgEG4, h3.080_IgEE, h1.194_IgEE, h1.041_IgEE, h3.152_IgEG4, h2.017_IgEE; T2 epitopes, in BF order: h2.008_IgEE, h2.008_IgEG4, h1.021_IgEE, h1.015_IgEE, h1.029_IgEE, h2.010_IgEG4, h1.194_IgEE, h1.186_IgEG4.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method for diagnosing a peanut allergy, and/or severity of a peanut allergy, in a subject comprising:
contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptides are coupled to a solid support, to form one or more AAI-peptide-solid support complexes;
binding an AAI-specific labeling reagent to the AAI-peptide-solid support complex; and
detecting binding of the AAI-specific labeling reagent to each AAI-peptide-solid support complex to identify one or more peanut peptides bound to the AAI in the serum or plasma of the subject;
wherein recognition of at least one peanut peptide by an AAI in the serum or plasma of the subject indicates that the subject is allergic to peanuts.

2. The method according to claim 1, wherein the one or more peanut peptides is derived from ara h 1 allergen (SEQ ID NO:1), ara h 2 allergen (SEQ ID NO:2), and/or ara h 3 allergen (SEQ ID NO:3).

3. The method according to claim 1, wherein the one or more peanut peptides is selected from:
peptides having at least 3 contiguous amino acids from positions 8 to 66 of ara h 1 allergen;
peptides having at least 3 contiguous amino acids from positions 103 to 152 of ara h 1 allergen;
peptides having at least 3 contiguous amino acids from positions 176 to 195 of ara h 1 allergen;
peptides having at least 3 contiguous amino acids from positions 5 to 40 of ara h 2 allergen;
peptides having at least 3 contiguous amino acids from positions 93 to 115 of ara h 3 allergen;
peptides having at least 3 contiguous amino acids from positions 30 to 75 of ara h 3 allergen; and/or
peptides having at least 3 contiguous amino acids from positions 152 to 167 of ara h 3 allergen.

4. The method according to claim 1, wherein the one or more peanut peptides comprise an amino acid sequence selected from any one or more of SEQ ID NOs: 4-67.

5-14. (canceled)

15. The method according to claim 1, wherein the determination that a subject is allergic to peanuts further takes into account the results of one or more of: total peanut specific IgE (sIgE), peanut component ara h 1 IgE, peanut component ara h 2 IgE, peanut component ara h 3 IgE, total peanut specific IgG4 (sIgG4), peanut component ara h 1 IgG4, peanut component ara h 2 IgG4, peanut component ara h 3 IgG4, skin prick test results, clinical or family history, and/or data from patient or clinician questionnaire.

16-17. (canceled)

18. The method according to claim 15, wherein the determination that a subject is allergic to peanuts further takes into account the results of one or more of:
peanut peptide comprising the amino acid sequence of SEQ ID NO:39 IgE, peanut peptide comprising the amino acid sequence of SEQ ID NO:45 IgG4, and peanut peptide comprising the amino acid sequence of SEQ ID NO:66 IgG4;
peanut peptide comprising the amino acid sequence of SEQ ID NO:39 IgE, peanut peptide comprising the amino acid sequence of SEQ ID NO:45 IgG4, and skin prick test;
peanut peptide comprising the amino acid sequence of SEQ ID NO:39 IgE, peanut peptide comprising the amino acid sequence of SEQ ID NO:45 IgG4, peanut peptide comprising the amino acid sequence of SEQ ID NO:66 IgG4, and sIgE; and
peanut peptide comprising the amino acid sequence of SEQ ID NO:39 IgE, peanut peptide comprising the amino acid sequence of SEQ ID NO:45 IgG4, peanut peptide comprising the amino acid sequence of SEQ ID NO:66 IgG4, and peanut component ara h 2.

19. The method according to claim 15, wherein the determination that a subject is allergic to peanuts comprises:
determining that the subject's sIgE is ≥0.03 kUA/L, which indicates that the subject may be allergic to peanuts, or determining that the subject's sIgE is <0.03 kUA/L, which indicates that the subject is not allergic to peanuts; and
when the subject's sIgE is ≥0.03 kUA/L, then determining whether the combination of the peanut peptide comprising the amino acid sequence of SEQ ID NO:39 IgE, peanut peptide comprising the amino acid sequence of SEQ ID NO:45 IgG4, and peanut peptide comprising the amino acid sequence of SEQ ID NO:66 IgG4 is <0.20, which indicates that the subject is not allergic to peanuts, or ≥0.20, which indicates that the subject is allergic to peanuts.

20-27. (canceled)

28. A method for detecting development of clinical tolerance to peanuts in a subject that is allergic to peanuts comprising:
contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptides are coupled to a solid support, to form one or more AAI-peptide-solid support complexes;
binding an AAI-specific labeling reagent to the AAI-peptide-solid support complex;
detecting binding of the AAI-specific labeling reagent to each AAI-peptide-solid support complex to identify one or more peanut peptides bound to the AAI in the serum or plasma of the subject; and
comparing the identified one or more peanut peptides bound to the AAI in the serum or plasma of the subject, or the concentration of the AAI in the serum or plasma of the subject, with a previously identified panel of one or more peanut peptides bound to the AAI in the serum or plasma of the subject, or a previous concentration of the AAI in the serum or plasma of the subject;
wherein development of clinical tolerance to peanuts is indicated when:
the subsequent number of peanut peptides recognized by IgE AAI in the serum or plasma of the subject, and/or the subsequent concentration of AAI IgE in the serum or plasma of the subject, is less than the previously identified number of peanut peptides recognized by AAI IgE in the serum or plasma of the subject, and/or less than the previous concentration of AAI IgE in the serum or plasma of the subject; and/or
the subsequent number of peanut peptides recognized by IgG4 AAI in the serum or plasma of the subject, and/or the subsequent concentration of AAI IgG4 in the serum or plasma of the subject, is greater than the previously identified number of peanut peptides recognized by AAI IgG4 in the serum or plasma of the subject, and/or greater than the previous concentration of AAI IgG4 in the serum or plasma of the subject.

29. The method according to claim 28, wherein the plurality of peanut peptides comprises at least two peptides derived from ara h 1 allergen (SEQ ID NO:1), ara h 2 allergen (SEQ ID NO:2), and/or ara h 3 allergen (SEQ ID NO:3).

30. The method according to claim 28, wherein the one or more peanut peptides is selected from:
peptides having at least 3 contiguous amino acids from positions 8 to 66 of ara h 1 allergen;
peptides having at least 3 contiguous amino acids from positions 103 to 152 of ara h 1 allergen;
peptides having at least 3 contiguous amino acids from positions 176 to 195 of ara h 1 allergen;
peptides having at least 3 contiguous amino acids from positions 5 to 40 of ara h 2 allergen;
peptides having at least 3 contiguous amino acids from positions 93 to 115 of ara h 3 allergen;
peptides having at least 3 contiguous amino acids from positions 30 to 75 of ara h 3 allergen; and/or
peptides having at least 3 contiguous amino acids from positions 152 to 167 of ara h 3 allergen.

31-41. (canceled)

42. The method of claim 28, wherein the determination that a subject is allergic to peanuts further takes into account the results of one or more of: total peanut specific IgE (sIgE), peanut component ara h 1 IgE, peanut component ara h2 IgE, peanut component ara h 3 IgE, skin prick test results, clinical or family history, and/or data from patient or clinician questionnaire.

43-50. (canceled)

51. A method for detecting an increase in intensity of allergy or adverse event during treatment of allergy to peanuts over time in a subject that is allergic to peanuts comprising:
contacting one or more peanut peptides with serum or plasma obtained from the subject under conditions sufficient to permit binding of one or more allergy associated immunoglobulins (AAIs) in the serum or plasma to the one or more peanut peptides, wherein the one or more peanut peptide are coupled to a solid support, to form one or more AAI-peptide-solid support complexes, and wherein the one or more peanut peptides is selected from peptides having at least 3 contiguous amino acids from positions 8 to 66 of ara h 1 allergen, peptides having at least 3 contiguous amino acids from positions 103 to 152 of ara h 1 allergen, peptides having at least 3 contiguous amino acids from positions 176 to 195 of ara h 1 allergen, peptides having at least 3 contiguous amino acids from positions 5 to 40 of ara h 2 allergen, peptides having at least 3 contiguous amino acids from positions 93 to 115 of ara h 3 allergen, peptides having at least 3 contiguous amino acids from positions 30 to 75 of ara h 3 allergen, and/or peptides having at least 3 contiguous amino acids from positions 152 to 167 of ara h 3 allergen;
binding an AAI-specific labeling reagent to the AAI-peptide-solid support complex;
detecting binding of the AAI-specific labeling reagent to each AAI-peptide-solid support complex to identify one or more peanut peptides bound to the AAI in the serum or plasma of the subject; and
comparing the identified one or more peanut peptides bound to the AAI in the serum or plasma of the subject, or the concentration of the AAI in the serum or plasma of the subject, with a previously identified panel of one or more peanut peptides bound to the AAI in the serum of the subject, or a previous concentration of the AAI in the serum or plasma of the subject;
wherein an increased intensity of the allergic response to peanuts is indicated when the subsequent number or pattern of reactivity of peanut peptides recognized by AAI in the serum or plasma of the subject, or the subsequent concentration of AAI in the serum or plasma of the subject, is greater than the previously identified number or pattern of reactivity of peanut peptides recognized by AAI in the serum or plasma of the subject, or greater than the previous concentration of AAI in the serum or plasma of the subject.

52. The method according to claim 51, wherein the one or more peanut peptides comprise an amino acid sequence selected from any one or more of SEQ ID NOs: 4-67.

53-62. (canceled)

63. The method of claim 51, wherein the determination that a subject is allergic to peanuts further takes into account the results of one or more of: total peanut specific IgE (sIgE), peanut component ara h 1 IgE, peanut component ara h2 IgE, peanut component ara h 3 IgE, skin prick test results, clinical or family history, and/or data from patient or clinician questionnaire.

64-71. (canceled)

72. A method of sensitizing an infant to one or more peanut allergens to induce tolerance or non-allergy to peanuts comprising administering one or more peanut peptides to the infant, wherein the one or more peanut peptides are derived from ara h 1 allergen (SEQ ID NO:1), ara h 2 allergen (SEQ ID NO:2), and/or ara h 3 allergen (SEQ ID NO:3).

73. The method according to claim 72, wherein the one or more peanut peptides is selected from:
peptides having at least 3 contiguous amino acids from positions 8 to 66 of ara h 1 allergen;
peptides having at least 3 contiguous amino acids from positions 103 to 152 of ara h 1 allergen;
peptides having at least 3 contiguous amino acids from positions 176 to 195 of ara h 1 allergen;
peptides having at least 3 contiguous amino acids from positions 5 to 40 of ara h 2 allergen;
peptides having at least 3 contiguous amino acids from positions 93 to 115 of ara h 3 allergen;
peptides having at least 3 contiguous amino acids from positions 30 to 75 of ara h 3 allergen; and/or
peptides having at least 3 contiguous amino acids from positions 152 to 167 of ara h 3 allergen.

74-84. (canceled)

85. A set of allergenic epitope-containing peanut peptides comprising a plurality of peanut peptides comprising at least two peptides derived from ara h 1 allergen (SEQ ID NO:1), ara h 2 allergen (SEQ ID NO:2), and/or ara h 3 allergen (SEQ ID NO:3).

86. The set of peanut peptides according to claim 85, wherein the plurality of peanut peptides is selected from:
peptides having at least 3 contiguous amino acids from positions 8 to 66 of ara h 1 allergen;
peptides having at least 3 contiguous amino acids from positions 103 to 152 of ara h 1 allergen;
peptides having at least 3 contiguous amino acids from positions 176 to 195 of ara h 1 allergen;
peptides having at least 3 contiguous amino acids from positions 5 to 40 of ara h 2 allergen;
peptides having at least 3 contiguous amino acids from positions 93 to 115 of ara h 3 allergen;
peptides having at least 3 contiguous amino acids from positions 30 to 75 of ara h 3 allergen; and/or
peptides having at least 3 contiguous amino acids from positions 152 to 167 of ara h 3 allergen.

87-97. (canceled)

98. A kit comprising:
a) one or more allergenic epitope-containing peanut peptides derived from ara h 1 allergen (SEQ ID NO:1), ara h 2 allergen (SEQ ID NO:2), and/or ara h 3 allergen (SEQ ID NO:3), wherein each peanut peptide is coupled to a solid support; and
b) an allergy associated immunoglobulin (AAI)-specific labeling reagent; packaged together and including instructions for use.

99-100. (canceled)

101. The kit according to claim 98, wherein the one or more peanut peptides is selected from:
peptides having at least 3 contiguous amino acids from positions 8 to 66 of ara h 1 allergen;
peptides having at least 3 contiguous amino acids from positions 103 to 152 of ara h 1 allergen;
peptides having at least 3 contiguous amino acids from positions 176 to 195 of ara h 1 allergen;
peptides having at least 3 contiguous amino acids from positions 5 to 40 of ara h 2 allergen;
peptides having at least 3 contiguous amino acids from positions 93 to 115 of ara h 3 allergen;
peptides having at least 3 contiguous amino acids from positions 30 to 75 of ara h 3 allergen; and/or
peptides having at least 3 contiguous amino acids from positions 152 to 167 of ara h 3 allergen.

102-117. (canceled)
